(19) United States
(12) Patent Application Publication
(10) Pub. No.: US 2023/0027353 A1
(43) Pub. Date: Jan. 26, 2023

(54) Systems and Methods for Deconvoluting Tumor Ecosystems for Personalized Cancer Therapy

(71) Applicant: The Board of Trustees of the Leland Stanford Junior University, Stanford, CA (US)

(72) Inventors: Arash Ash Alizadeh, San Mateo, CA (US); Aaron M. Newman, San Mateo, CA (US); Chloe B. Steen, Oslo (NO); Bogdan Luca, Stanford, CA (US); Andrew J. Gentles, Palo Alto, CA (US)

(73) Assignee: The Board of Trustees of the Leland Stanford Junior University, Stanford, CA (US)

(21) Appl. No.: 17/755,713

(22) PCT Filed: Nov. 5, 2020
(86) PCT No.: PCT/US20/59196

§ 371 Date: May 5, 2022

Related U.S. Application Data
(60) Provisional application No. 62/931,047, filed on Nov. 5, 2019.

(51) Int. Cl. G16H 20/10; G16B 25/10; G16B 40/20; G16H 10/40

(52) U.S. Cl. CPC G16H 20/10; G16B 25/10; G16B 40/20; G16H 10/40

(57) ABSTRACT

Methods and systems for deconvoluting tumor ecosystems for personalized cancer therapy are disclosed. Generally, human cancers exhibit large variation in behavior between and within patients, which is in large part related to cellular composition. Identifying cell types can identify specific types of tumors and/or cancers present in an individual. Further embodiments generally describe identifying therapies from clinical trials to which the tumor or cancer ecotypes respond, thus providing personalized therapies based on the identified cancer or tumor type.

This application claims priority to U.S. Provisional Application Ser. No. 62/931,047, entitled “Systems and Methods for Deconvoluting Tumor Ecosystems for Personalized Cancer Therapy” to Alizadeh et al., filed Nov. 5, 2019; the disclosure of which is herein incorporated by reference in its entirety.

### FIELD OF THE INVENTION

The present application relates generally to personalized medicine and more specifically to personalized therapies for cancers based on tumor ecotype.

### BACKGROUND

Human cancers exhibit large variation in behavior between and within patients, which is in large part related to cellular composition. For example, diffuse large B cell lymphoma (DLBCL) exhibits significant clinical and biological heterogeneity, in part due to cell-of-origin subtypes, somatic alterations, and diverse stromal constituents within the tumor microenvironment (TME). Several immunologically-active lymphoma therapies are known to rely on innate and adaptive anti-tumor responses occurring within this dynamic TME, including agents that are approved (e.g., rituximab, lenalidomide, CART19, ibrutinib) or emerging (e.g., anti-CD47, checkpoint inhibitors). No work has shown that a large-scale characterization of the cellular heterogeneity in DLBCL will reveal previously unknown biological variation in the TME linked to tumor subtypes and genotypes, therapeutic responses, and clinical outcomes, with implications for future personalization of immunotherapy.

### SUMMARY OF THE INVENTION

Methods and systems for deconvoluting tumor ecosystems for personalized cancer therapy are disclosed.

In one embodiment, a method for treating an individual for a tumor includes obtaining gene expression data from a tumor obtained from an individual, characterizing a tumor ecosystem for the tumor based on the gene expression data, where the tumor ecosystem is comprised of spatially and temporally-linked cell states, identifying an efficacious treatment for the tumor based on clinical treatment data, where the clinical treatment data identifies at least one treatment shown to be efficacious for a tumor exhibiting the tumor ecosystem, and treating the individual with the efficacious treatment for the tumor.

In a further embodiment, the characterizing a tumor ecosystem step includes purifying a gene expression profile of cell types within the tumor, identifying at least one cell state in the tumor based on the gene expression profiles, and identifying the tumor ecosystem based on the at least one cell state.

In another embodiment, the identifying the tumor ecosystem step comprises using a trained negative matrix factorization (NMF) model to identify the tumor ecosystem.

In a still further embodiment, the NMF model is trained by obtaining cellular expression data from a plurality of samples from one or more tissue types, purifying gene expression profiles of cell types within plurality of samples based on the cellular expression data, identifying cell states of the cell types by clustering cell type-specific gene expression profiles, and classifying the plurality of samples into tumor ecosystem subtypes by identifying cell states that co-occur in the same sample.

In still another embodiment, the purifying step uses a digital cytometry algorithm for to purify the gene expression profiles.

In a yet further embodiment, the digital cytometry algorithm is CIBERSORTx.

In yet another embodiment, the one or more tissue types include at least one cancer or tumor.

In a further embodiment again, the at least one cancer or tumor is selected from the group consisting of: lymphomas and carcinomas.

In another embodiment again, the at least one cancer or tumor is selected from the group consisting of: diffuse large B cell lymphoma, -small cell lung cancer, breast cancer, colorectal cancer, and head and neck squamous cell carcinoma.

In a further additional embodiment, the cellular expression data is obtained from single cell RNA sequencing.

In another additional embodiment, the NMF model is employed via Kullback-Leibler divergence minimization.

In a still yet further embodiment, the identifying cell states calculate a cophenetic coefficient for a range of cluster numbers as part of clustering.

In still yet another embodiment, the clustering further comprises filtering to remove low quality cell states.

In a still further embodiment again, the filter removes cell states with fewer than 10 genes.

In still another embodiment again, the filter removes cell states with low levels of expression.

In a still further additional embodiment, the NMF model training further comprises updating the NMF model by iteratively updating the model until convergence.

In still another additional embodiment, the at least one treatment is selected from chemotherapeutics, immunotherapeutics, radiation, and combinations thereof.

In a yet further embodiment again, the method further includes obtaining a tumor sample or a cancer sample from an individual, wherein the gene expression data is obtained from the tumor sample or the cancer sample.

In yet another embodiment again, the tumor sample or the cancer sample is obtained from a biopsy.

In a yet further additional embodiment, the gene expression data is obtained from RNA sequencing, single cell RNA sequencing, or a microarray.

### BRIEF DESCRIPTION OF THE DRAWINGS

FIG. 1 illustrates a method for training a model to identify tumor microenvironments in accordance with various embodiments of the invention.

FIG. 2 illustrates a method to treat an individual based on a tumor microenvironment in accordance with various embodiments of the invention.

FIG. 3A illustrates a schematic of an embodiment application to DLBCL. 522 DLBCL tumor biopsies profiled by RNA-seq were digitally purified with CIBERSORTx (into cell-specific gene expression profiles of 13 cell types. EcoTyper was then applied to the digitally-purified cell gene expression profiles to identify distinct transcriptional cell states. These were next interrogated in scRNA-seq atlases and independent DLBCL patient cohorts, and associated with overall survival. Finally, EcoTyper defined cellular communities that constitute lymphoma ecosystem subtypes, or “lymphoma ecotypes”.

FIG. 3B illustrates an overview of patient cohorts analyzed for discovery and recovery of DLBCL cell states and lymphoma ecotypes in accordance with various embodiments of the invention.

FIG. 3C illustrates a UMAP plot of scRNA-seq of 2 DLBCL, 3 FL and 1 tonsil scRNA-seq dataset generated in accordance with various embodiments of the invention.

FIG. 3D illustrates an overview of lymphoid scRNA-seq atlases for recovery of cell states identified in accordance with various embodiments of the invention.

FIG. 4A illustrates a heat map depicting the relative log₂ gene expression of top marker genes in 5 transcriptional cell states in accordance with various embodiments of the invention.

FIG. 4B illustrates a heat map depicting the relative log₂ gene expression of the same genes and cell states shown in FIG. 4A in independent DLBCL cohorts profiled by microarray and RNA-seq of fresh-frozen and formalin-fixed tissues in accordance with various embodiments of the invention.

FIG. 4C illustrates recover of defined B cell state and annotated with cell-of-origin subtype information in accordance with various embodiments of the invention.

FIG. 4D illustrates concordance of B cell state composition in ABC and GCB DLBCL samples profiled by gene expression profiling of bulk samples (left) and single cells (right) in accordance with various embodiments of the invention.

FIG. 4E illustrates a comparison of Lymphgen mutational subtype sample annotation and dominant B cell states in accordance with various embodiments of the invention.

FIG. 5A illustrates a UMAP plot of derived transcriptional cell states of the 12 cell types of the DLBCL tumor microenvironment in the discovery cohort in accordance with various embodiments of the invention.

FIG. 5B illustrates a heat map depicting relative log₂ expression of marker genes across T cell types and 14 cell states in the discovery cohort (left) and six scRNA-seq atlases (right) in accordance with various embodiments of the invention.

FIG. 5C illustrates survival associations of TME cell states across four DLBCL patient cohorts in accordance with various embodiments of the invention.

FIG. 6A illustrates concordance of cell states skewed towards ABC or GCB DLBCL in 4 DLBCL patient cohorts and five DLBCL scRNA-seq samples in accordance with various embodiments of the invention.

FIG. 6B illustrates a Kaplan-Meier plot showing differences in overall survival between patients with DLBCL tumors assigned to a dominant cell state significantly enriched in GCB DLBCL or ABC DLBCL in accordance with various embodiments of the invention.

FIG. 6C illustrates heat maps showing the co-occurrence of cell state abundance profiles in ABC and GCB DLBCL in the discovery cohort, organized by distinct cell communities defined by hierarchical clustering in accordance with various embodiments of the invention.

FIG. 7A illustrates a distribution of cell state abundances across 473 DLBCL samples assigned to nine lymphoma ecotypes in accordance with various embodiments of the invention.

FIG. 7B illustrates network diagrams depicting cell states organized into nine lymphoma ecotypes in accordance with various embodiments of the invention.

FIG. 8A illustrates a schematic of workflow for analysis of the REMoDL-B clinical trial gene expression dataset with EcoTyper in accordance with various embodiments of the invention.

FIG. 8B illustrates an overview of cell states associated with overall survival in the RB-CHOP arm relative to the R-CHOP arm and their LE membership in accordance with various embodiments of the invention.

FIG. 9 illustrates a schematic depicting the EcoTyper framework and its application to carcinoma in accordance with various embodiments of the invention.

FIG. 10A illustrates heat maps showing digitally-purified expression profiles of 12 cell types decoded from 16 bulk epithelial tumor types by CIBERSORTx, with genes as rows and tumor/adjacent normal tissue samples as columns in accordance with various embodiments of the invention.

FIG. 10B illustrates a UMAP projection of FIG. 10A in accordance with various embodiments of the invention.

FIG. 10C illustrates heat maps depicting the expression of cell state-specific marker genes across seven scRNA-seq datasets spanning four types of carcinoma in accordance with various embodiments of the invention.

FIG. 10D illustrates enrichment of EcoTyper states in normal adjacent tissue (Chi-square test), comparing the discovery cohort, which was digitally purified from bulk RNA-seq, to EcoTyper states recovered from an scRNA-seq tumor atlas in accordance with various embodiments of the invention.

FIG. 10E illustrates H&E staining of colorectal cancer specimens and analysis of monocyte/macrophage marker genes in bulk RNA-seq profiles of laser micro-dissected stroma in accordance with various embodiments of the invention.

FIG. 11A illustrates survival associations of 69 cell states in 5,946 tumors, stratified by cell type and aggregated across malignancies using Stouffer's method in accordance with various embodiments of the invention.

FIG. 11B illustrates state-specific survival associations in the discovery cohort (TCGA) and an independent cohort of >9,000 epithelial tumor transcriptomes (PRECOG) in accordance with various embodiments of the invention.

FIG. 12A illustrates cell-state abundance profiles across 16 carcinomas rendered as a heat map, in which cell states are organized into 10 multicellular communities, called carcinoma ecotypes in accordance with various embodiments of the invention.

FIG. 12B illustrates network diagrams of CE-specific cell types and states in accordance with various embodiments of the invention.

FIG. 12C illustrates a schematic overview of the CE recovery approach in accordance with various embodiments of the invention.

FIG. 12D illustrates heat maps portraying co-occurrence relationships among cell state abundance profiles, both in the TCGA discovery cohort (left) and in a validation cohort consisting of five scRNA-seq tumor atlases spanning NSCLC, CRC, breast cancer, and HNSC (right) in accordance with various embodiments of the invention.

FIG. 13A illustrates characteristics of carcinoma ecotypes (CEs) in the discovery cohort in accordance with various embodiments of the invention.

FIG. 13B illustrates CE composition and pan-carcinoma survival associations in normal tissues (GTEx) and primary tumor and adjacent normal (TCGA) samples from the discovery cohort in accordance with various embodiments of the invention.

FIG. 13C illustrates an association of 118 features with overall survival and response to ICI in 571 patients with advanced melanoma (Mel.) or bladder cancer (BLCA) in accordance with various embodiments of the invention.

FIG. 14A illustrates heat maps displaying differentially expressed genes between CE9 and CE10 in seven scRNA-seq tumor datasets, shown for cell types that are present in both carcinoma ecotypes in accordance with various embodiments of the invention.

FIG. 14B illustrates three-channel immunofluorescence imaging of DAPI, CD3, and either GZMK (top) or GZMB (bottom) in non-small cell lung cancer (NSCLC) specimens with paired RNA-seq in accordance with various embodiments of the invention.

FIG. 14C illustrates a distribution of CE9 and CE10 in breast and melanoma tumor sections profiled on spatial transcriptomics arrays (left) and relative distance of CE9- and CE10-positive spots (right) from epithelial cells (top) and melanoma cells (bottom) in accordance with various embodiments of the invention.

FIG. 14D illustrates heat maps depicting Spearman cross-correlation matrices of TME cell states from CE9 and CE10 across barcoded spots in spatial transcriptomics arrays of breast cancer specimens (n=2 sections, 1 patient) and melanoma specimens (n=8 sections, 4 patients) in accordance with various embodiments of the invention.

FIG. 14E illustrates a schema illustrating clinical outcomes of 33 subjects for whom premalignant lung lesions were profiled by microarray and assessed for CE9 and CE10 by EcoTyper (left) and box plots showing the relative abundance of CE9 versus CE10 in premalignant lung lesions, stratified by clinical outcome (right) in accordance with various embodiments of the invention.

### DETAILED DESCRIPTION

Turning now to the drawings, systems and methods for deconvoluting tumor ecosystems for personalized cancer therapy are illustrated. Tumors are complex ecosystems consisting of malignant, immune, and stromal elements whose dynamic interactions drive patient survival and response to therapy. Tumor ecosystems are generally comprised of comprised of spatially and temporally-linked cell states. The advent of single cell RNA-sequencing (scRNA-seq) have enabled whole-transcriptional surveys of cell subsets at single cell level in lymphomas, dissecting the expression of checkpoint molecules on lymphoma-associated T cells, and showing the impact of tumor subclonal transcriptional heterogeneity on drug response. (See e.g., Andor, N., et al. (2019). Single-cell RNA-Seq of follicular lymphoma reveals malignant B-cell types and coexpression of T-cell immune checkpoints. Blood 133, 1119-1129; Aoki, T., et al. (2020). Single-Cell Transcriptome Analysis Reveals Disease-Defining T-cell Subsets in the Tumor Microenvironment of Classic Hodgkin Lymphoma. Cancer Discov 10, 406-421; and Roider, T., et al. (2020). Dissecting intratumour heterogeneity of nodal B-cell lymphomas at the transcriptional, genetic and drug-response levels. Nat Cell Biol 22, 896-906; the disclosures of which are hereby incorporated by reference in their entireties.) Although providing critical insights into the clinically-relevant cellular diversity of lymphomas, scRNA-seq studies so far have been of moderate size (less than 30 samples), and may be prone to dissociation distortions and patient-specific heterogeneity, making it challenging to identify prognostic cell states and ecosystems that are generalizable across patients.

A comprehensive understanding of the diversity of cellular states within the tumor microenvironment (TME), and their patterns of co-occurrence, could provide new diagnostic tools for improved disease management and novel targets for therapeutic intervention. To address this challenge, many embodiments describe a novel machine learning framework for large-scale identification of TME cell states and their co-association patterns from bulk, single-cell, and spatially resolved tumor expression data.

Various embodiments employ a computational framework to derive a high resolution cell atlas across tumor cell types. In some embodiments the cell types are purified from tumors or cancers, including (but not limited to) lymphomas and carcinomas. Various embodiments purify cell types from diffuse large B cell lymphoma (DLBCL) and carcinomas, including (but not limited to) non-small cell lung cancer, breast cancer, colorectal cancer, and head and neck squamous cell carcinoma. In certain embodiments, certain cell categories are dissected into distinct cell states.

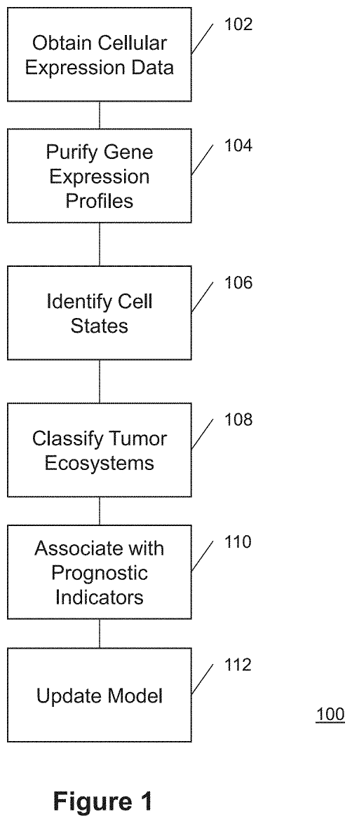

Turning to FIG. 1, a method 100 in accordance with many embodiments to train a model for TME identification. At 102, many embodiments obtain cellular expression data from a plurality of samples from one or more tissue types. In certain embodiments the tissue is cancer and/or tumor tissue, while some embodiments obtain healthy tissue. Certain embodiments obtain a combination of healthy and diseased tissue (e.g., a mix of cancer/tumor and healthy tissue). Various embodiments obtain the expression data by performing single cell RNA sequencing (scRNA-seq), while some embodiments obtain the scRNA-seq data directly, such as from a public or private database, including The Cancer Genome Atlas (TCGA). (See e.g., Tatlow, P. J., and Piccolo, S. R. (2016). A cloud-based workflow to quantify transcript-expression levels in public cancer compendia. Sci Rep 6, 39259; the disclosure of which is hereby incorporated by reference in its entirety.) Certain embodiments both perform scRNA-seq on tissue and obtain scRNA-seq data. However, many embodiments obtain batch RNA sequencing data, where the entire RNA of the tissue is obtained, either through sequencing tissue or by obtaining sequencing data from already sequenced tissue. Various embodiments obtain cellular expression data from a plurality of individuals or specimens. Some embodiments obtain cellular expression data from more than 100, 200, 300, 500, 1,000, or more samples.

At 104, various embodiments purify gene expression profiles of cell types. Various embodiments use an in silico cytometry algorithm to purify the gene expression profiles. Some embodiments use CIBERSORTx, a recently described machine learning platform for digital cytometry, as the in silico cytometry algorithm. (See e.g., Newman, A. M., et al. (2019). Determining cell type abundance and expression from bulk tissues with digital cytometry. Nat Biotechnol 37, 773-782; the disclosure of which is hereby incorporated by reference in its entirety.) CIBERSORTx minimizes technical variation across platforms and can simultaneously purify expression profiles from multiple cell types (>10) at single-sample resolution. As input, CIBERSORTx requires a collection of optimized expression profiles that discriminate each cell type of interest, commonly termed a ‘signature matrix’. Signature matrices can be derived from single-cell or bulk-sorted transcriptomes and should be designed to cover major lineages within a particular tissue type. The following equations and goals summarize the key CIBERSORTx steps used by EcoTyper:

B×F_{•,j}=M′_{•,j}, 1≤j≤n (1)

diag(G_{i,•,•}×F)=M_{i,•}, 1≤i≤g (2)

Given B, an m×c signature matrix consisting of m marker genes by c distinct cell types, and M′, an m×n matrix of bulk tissue gene expression profiles consisting of the same m genes by n samples, the goal of Equation 1 is to impute F, a c×n matrix consisting of the fractional abundances of c cell types for each sample in M′. (Note that M_{1,•} and M_{•,j} denote row i and column j of matrix M, respectively). Once F is imputed, the goal of Equation 2, which summarizes the high-resolution expression purification step of CIBERSORTx, is to impute G, a g×n×c matrix consisting of g genes, n samples, and c cell types, given F and M.

At 106, many embodiments identify distinct transcriptional programs, or “cell states,” upregulated in each cell type by clustering cell type-specific gene expression profiles. Many embodiments use a non-negative matrix factorization (NMF), or variant thereof, to identify transcriptional cell states in purified gene expression profiles. For example, Given c cell types, let Vᵢ←G_{•,•,i} be a g×n cell type-specific expression matrix for cell type i consisting of g rows (the number of genes) and n columns (the number of samples). The primary objective of NMF is to factorize Vᵢ into two non-negative matrices: a g×k matrix, W, and a k×n matrix, H, where k is a user-specified rank (i.e., number of clusters):

Vᵢ=W×H (3)

Some embodiments employ NMF via Kullback-Leibler (KL) divergence minimization, which starts with random initializations of the W and H matrices. (See e.g., Brunet, J. P., et al. (2004). Metagenes and molecular pattern discovery using matrix factorization. Proc Natl Acad Sci USA 101, 4164-4169; the disclosure of which is hereby incorporated by reference in its entirety.) This approach iteratively updates the following two equations until KL divergence is minimized:

$${W}_{gk} ← {W}_{gk} \frac{{Σ}_{n} [ {H}_{kn} {V}_{gn} / {( W \times H )}_{gn} ]}{{Σ}_{n} {H}_{kn}} \qquad ( 4 )$$

$${H}_{kn} ← {H}_{kn} \frac{{Σ}_{g} [ {W}_{gk} {V}_{gn} / {( W \times H )}_{gn} ]}{{Σ}_{g} {W}_{gk}} \qquad ( 5 )$$

Here, each cluster corresponds to a cell state. The basis matrix, W, encodes a representative expression level for each gene in each cell state. The mixture coefficients matrix H encodes the representation (relative abundance) of each cell state in each sample. Compared to alternative clustering approaches, NMF has three main advantages for cell-state discovery from digitally-purified transcriptomes. First, NMF naturally decomposes each expression profile into a set of constituent states. This sample-level decomposition is appropriate since purified expression profiles are akin to bulk-sorted populations, which may contain multiple cell states in a given sample. Second, NMF identifies a set of states that best explain all purified expression profiles (for a given cell type) while simultaneously quantifying the relative abundance of each cell state. Third, NMF has analytical properties that enable assignment and validation of cell states in new data without re-training the model or deriving another classifier.

Some embodiments apply NMF independently to each digitally-purified expression matrix Vᵢ. In some embodiments, cell types with >1,000 detectably expressed genes, the top 1,000 genes with highest relative dispersion are selected as input. To do this for a given expression matrix Vᵢ, genes in log₂ space can be averaged across samples and binned into groups (e.g., 20 groups by 5 percentile increments). The relative dispersion of each gene was then calculated as the difference between its dispersion and the median dispersion of genes within the same expression bin, divided by the median absolute deviation of the dispersion of genes within the same expression bin.

As part of the clustering procedure, certain embodiments calculate a cophenetic coefficient for a range of cluster numbers, which can help determine the most stable number of cell states for each cell type. Some embodiments select a number of clusters closest to a cophenetic coefficient of 0.99. Some embodiments apply one or more filters to remove low-quality cell states. One possible filter removes cell states with very few marker genes (e.g., fewer than 10 genes). A second possible filter calculates a posneg ratio filter, which removes cell states with low levels of expression and most likely to represent low-quality cell states. Some embodiments output the sample cell states as a mixture of cell states, while certain embodiments assign a sample to a discrete cell state where the most dominant cell state in a given sample is assigned.

At 108, certain embodiments classify tumors into tumor ecosystem subtypes by identifying cell states that co-occur in the same sample. Various embodiments refer to the tumor ecosystem subtypes as “tumor ecotypes.” Some embodiments leverage a Jaccard index to calculate the overlap between pairs of cell states to identify subtypes. To this end, certain embodiments discretize each cell state q into a binary vector a of length l, where l=the number of tumor samples in the discovery cohort. Collectively, these vectors comprised binary matrix A, with 69 rows (states)×l columns (samples). Given sample s, if state q was the most abundant state among all states in cell type i, we set A_{q,s} to 1; otherwise A_{q,s}←0. We then computed all pairwise Jaccard indices on the rows (states) in matrix A, yielding matrix J with a number of rows and columns equal to the number of cell states identified in these embodiments (e.g., if 20 cell states are identified, the matrix has dimensions of 20 rows×20 columns). Additional embodiments employ a hypergeometric test to evaluate the null hypothesis that any given pair of cell states q and k has no overlap. In cases where the hypergeometric p-value was >0.01, the Jaccard index for J_{q,k} is set to 0 (i.e., no overlap). To identify communities while accommodating outliers, the updated Jaccard matrix J′ is hierarchically clustered using average linkage with Euclidean distance (hclust in the R stats package) in certain embodiments. The optimal number of clusters can then be determined via silhouette width maximization.

In many embodiments, the tumor ecosystems are associated with prognostic indicators at 110. Prognostic indicators include survival, therapeutic response, and/or any other indicator that has been identified based on the origination of the samples from which cellular expression data is initially obtained. As such, some embodiments are able to improve medical technologies by identifying specific therapies or outlooks for specific tumor ecosystems that exist within one cancer. In some embodiments, the prognostic indicators are stored as metadata along with tumor ecosystems identified within the model.

At 112, various embodiments update the model with new samples. In classical NMF, matrices W and H are iteratively updated according to Equations 4 and 5 until convergence. However, various embodiments introduce a new dataset (e.g., gene expression data), V′, and reuse a previously fit cell type-specific basis matrix W in order to determine the mixture coefficients matrix H′ in new samples:

V′=W×H′ (6)

This update procedure consists of iteratively updating H′ until convergence of Equation 6. This approach has three distinct advantages over alternative methods for supervised classification. First, the mathematical structure of the original model is maintained when classifying new samples. This eliminates the need to train another classifier and avoids the introduction of new assumptions or biases that lead to information loss. Second, this approach mirrors the output of the original NMF model, facilitating consistent interpretation. Third, unlike methods that perform supervised classification independently for each sample, the matrix H′ is jointly updated across all samples, increasing the robustness of cell state recovery.

It should be noted that the various features illustrated in reference to method 100 may be omitted, performed in a different order (including simultaneously), and/or repeated as applicable to certain embodiments. For example, if an embodiment does not introduce additional data, updating a model 112 would not be included in that particular embodiment. Additionally,

### Methods of Treating an Individual

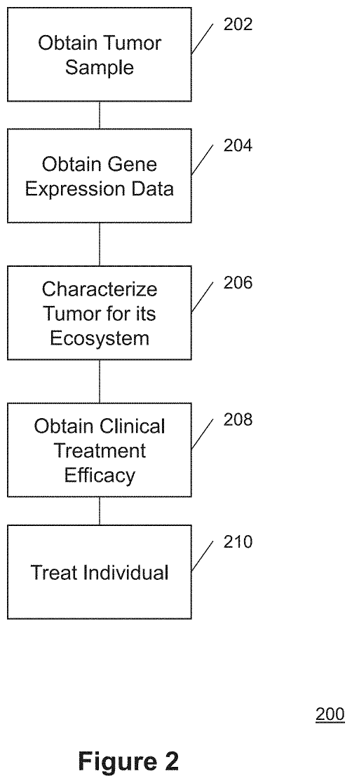

Turning to FIG. 2, a method 200 to treat an individual based on a tumor ecosystem is illustrated. Many of these embodiments obtain a tumor and/or cancer sample from an individual at 202. In various embodiments the tumor sample is a biopsy of a tumor, including (but not limited to) fine needle aspiration biopsy, core needle biopsy, vacuum-assisted biopsy, excisional biopsy, shave biopsy, punch biopsy, endoscopic biopsy, laparoscopic biopsy, bone marrow aspiration and biopsy, liquid biopsy, and/or a combination thereof.

At 204, many embodiments obtain gene expression data from the sample. Various embodiments obtain the gene expression data from RNA sequencing, including scRNA-seq, whole tissue RNA sequencing, microarray data, and/or any other form of expression data.

Additional embodiments characterize the tumor for its tumor ecosystem at 206. In many of these embodiments, the tumor ecosystem is characterized by dissecting the cell types and identifying the tumor ecosystem, such as described above in relation to method 100, where cell lineages, cell types, and tumor ecosystems are determined via a trained NMF model.

At 208, certain embodiments associate the identified tumor ecosystem with clinical treatment efficacy and/or prognostics for a disease (e.g., cancer and/or tumor) based on clinical data. In various embodiments, the clinical treatment data involves clinical trials for a particular type of tumor (e.g., lymphoma, carcinoma, etc.). In many of these embodiments, tumor ecosystem subtypes of the individuals in the clinical study are obtained and correlated to the efficacy of a particular treatment (e.g., drug, therapy, etc.). In some embodiments, the prognostic indicator and/or treatment is obtained along with the tumor ecosystem, as metadata from a model.

At 210, many embodiments apply the treatment identified by efficacy to the individual to treat the disease. In many embodiments, the treatment is selected from chemotherapeutics, immunotherapeutics, radiation, any other known or discovered treatment for a particular cancer and/or tumor, and any combination thereof.

It should be noted that the various features illustrated in reference to method 200 may be omitted, performed in a different order (including simultaneously), and/or repeated as applicable to certain embodiments. For example, some embodiments may simultaneously obtain clinical treatment data with the characterization of the tumor ecosystem of the individual.

### EXEMPLARY EMBODIMENTS

Although the following embodiments provide details on certain embodiments of the inventions, it should be understood that these are only exemplary in nature, and are not intended to limit the scope of the invention.

### Example 1: The Landscape of Tumor Cell States and Cellular Ecosystems in Diffuse Large B Cell Lymphoma

BACKGROUND: Diffuse large B cell lymphoma (DLBCL) is a cancer type that arises from B lymphocytes and that exhibits significant clinical and biological heterogeneity. Although the major clinical distinction in DLBCL is based on its cell of origin, where patients with germinal center B cell-like (GCB) DLBCL show longer survival compared to patients with activated B cell-like (ABC) DLBCL, signatures reflecting the DLBCL tumor microenvironment (TME) have also been shown to be significantly prognostic. Indeed, several immune-activating therapies, such as the use of monoclonal antibodies, checkpoint blockade and chimeric antigen receptor T cells, have been approved or are currently being investigated for treatment of DLBCL. However, DLBCL remains incurable for approximately 40% of patients, and a better understanding of the DLBCL TME could help identify more effective therapies.

More recently, the advent of single cell RNA-sequencing (scRNA-seq) have enabled whole-transcriptional surveys of cell subsets at single cell level in lymphomas, dissecting the expression of checkpoint molecules on lymphoma-associated T cells, and showing the impact of tumor subclonal transcriptional heterogeneity on drug response. Although providing critical insights into the clinically-relevant cellular diversity of lymphomas, scRNA-seq studies so far have been of moderate size (less than 30 samples), and may be prone to dissociation distortions and patient-specific heterogeneity, making it challenging to identify prognostic cell states and ecosystems that are generalizable across patients. Furthermore, the transcriptional states of the DLBCL TME remain undefined, and a large-scale analysis of the DLBCL TME and its clinical relevance is currently lacking.

This embodiment employed a computational framework, referred to as EcoTyper, to derive a high-resolution cell atlas across 13 cell types digitally purified from 522 DLBCL tumors. This embodiment dissected the B cell compartment of DLBCL into five distinct cell states. These B cell states are ubiquitous across 1,050 independent DLBCL tumors and 12,000 B cells profiled by scRNA-seq and exhibit major differences in prognosis and tumor specificity. Next, this embodiment demonstrated that the TME plays a critical role in DLBCL clinical outcomes, with eight TME cell states being more prognostic than the most favorable B cell state, and seven of these are prognostic independently of cell-of-origin. Finally, we describe how cell states form distinct tumor ecosystems that extend beyond the traditional cell-of-origin and mutational classification of DLBCL. Together, the findings provide an unprecedented systems-level portrait of the prognostic tumor microenvironment and ecosystems in DLBCL.

### Methods:

### Bulk Tumor Datasets

The dataset described in the study by Schmitz and colleagues (Schmitz et al., 2018) was selected as discovery cohort, and was downloaded from the website of the National Cancer Institute (NCI). (See Schmitz, R., et al. (2018). Genetics and Pathogenesis of Diffuse Large B-Cell Lymphoma. N Engl J Med 378, 1396-1407; the disclosure of which is hereby incorporated by reference in its entirety.) The samples from the Cancer Genome Atlas (TCGA) were excluded (n=40) due to batch effects. The gene expression values were normalized to transcripts per million (TPM). All 522 RNA-seq samples were included for defining cell states and ecosystems using EcoTyper.

The validation cohorts consist of three DLBCL datasets from prior studies. The raw Affymetrix CEL files of the cohort by Chapuy and colleagues were obtained from GEO (GSE98588), and processed using a custom chip definition file (cdf v23), as previously described. (See Chapuy, B., et al. (2018). Molecular subtypes of diffuse large B cell lymphoma are associated with distinct pathogenic mechanisms and outcomes. Nat Med 24, 679-690; and Newman, A. M., et al. (2015). Robust enumeration of cell subsets from tissue expression profiles. Nat Methods 12, 453-457; the disclosures of which are hereby incorporated by reference in their entireties.) The gene expression matrix file of the cohort by Ennishi and colleagues was kindly shared by the authors. (See Ennishi, D., et al. (2019). Double-Hit Gene Expression Signature Defines a Distinct Subgroup of Germinal Center B-Cell-Like Diffuse Large B-Cell Lymphoma. J Clin Oncol 37, 190-201; the disclosure of which is hereby incorporated by reference in its entirety.) The count matrix was gene-length-normalized and then normalized to TPM. The gene expression matrix from the cohort by Reddy and colleagues was kindly shared by the authors. (See Reddy, A., et al. (2017). Genetic and Functional Drivers of Diffuse Large B Cell Lymphoma. Cell 171, 481-494 e415; the disclosure of which is hereby incorporated by reference in its entirety.) For each validation cohort, the clinical data was obtained from the corresponding publication. The cell-of-origin labels provided by the authors of each respective study were used for enrichment analyzes. The LymphGen labels from the study by Wright and colleagues defined for the discovery cohort and two of the validation cohorts were used for enrichment analyzes. (See Wright, G. W., et al. (2020). A Probabilistic Classification Tool for Genetic Subtypes of Diffuse Large B Cell Lymphoma with Therapeutic Implications. Cancer Cell 37, 551-568 e514; the disclosure of which is hereby incorporated by reference in its entirety.)

To identify a biomarker for response to the drug bortezomib the gene expression matrix from the REMoDL-B trial from GEO (GSE117556) was obtained and the corresponding clinical data from the supplement of the study by Sha and colleagues. (See Sha, C., et al. (2019). Molecular High-Grade B-Cell Lymphoma: Defining a Poor-Risk Group That Requires Different Approaches to Therapy. J Clin Oncol 37, 202-212; the disclosure of which is hereby incorporated by reference in its entirety.)

Processing of scRNA-Seq Dataset Generated for this Study

Cell suspensions were obtained from patients diagnosed with DLBCL (n=2; one ABC-DLBCL and one GCB-DLBCL), FL (n=3) and tonsillitis (n=1; normal control). The samples were thawed, and 100,000 live cells were sorted by flow cytometry using two antibody markers specific for B cells, CD19 (BioLegend Cat #363023, RRID:AB_2564252; BioLegend Cat #363035, RRID:AB_2632786) and CD20 (BD Biosciences Cat #641396, RRID:AB_1645724), in addition to a live-dead marker (Aqua live-dead, Thermofisher, Cat #L34965). Two mutually exclusive populations were sorted, a CD19+ and CD20+ positive B cell population, and a CD19− and CD20− non-B cell population. The sorted populations were resuspended in FACS buffer (phosphate buffered saline with 5% fetal calf serum blocking buffer). The samples were processed for scRNA-seq library preparation at the Stanford Functional Genomics Facility immediately after FACS sorting with the 10× Chromium 5′ kit (10× Genomics, Pleasanton, Calif.) and the 10× Chromium Single Cell Human BCR Amplification kit, following the manufacturer's protocol. The targeted number of captured cells was 3,000 cells. Sequencing was performed on a HiSeq 4000 (IIlumina, Inc., San Diego, Calif.). Samples sorted and sequenced at the same time were combined on the same sequencing lane to avoid technical batch effects. scRNA-seq and scVDJ-seq of the B cell samples were sequenced together. The resulting scRNA-seq raw sequencing data was processed with the CellRanger pipeline (version 2.1 and 3.0, 10× Genomics) and mapped to the hg19 reference genome, resulting in gene expression count matrices with genes as rows and cell barcodes as columns. The scVDJ-seq raw sequencing data were mapped to reference “refdata-cellranger-vdj-GRCh38-alts-ensembl-4.0.0”. The final clonotypes were downloaded from the Loupe VDJ browser v3.0.0 (10× Genomics).

Cell Annotation of scRNA-Seq Dataset Generated for this Study

Seurat (v3.0) was used to process and annotate cell types. The Cell Ranger output files for the DLBCL samples were first each analyzed separately in Seurat to remove low-quality cells. After pre-processing, the cell types were then annotated in all four samples together (B cells and non-B cells samples for each DLBCL case), with clustering resolution parameter of 1.2 and using 20 dimensions. B cells were labeled based on expression of MS4A1 and CD79B, T cells based on expression of CD3D and CD3E, with expression of CD8B, CD8A and CD4 used to distinguish CD8 and CD4 T cells. Follicular helper T cells were defined as the cluster showing high expression of CXCL13, regulatory T cells as high expression of FOXP3, myeloid cells by expression of CD14, FCER1A, FCGR3A and NKs by expression of GLNY and NKG7. The FL and tonsil samples were analyzed each sample individually, and annotated using the same set of genes as listed above.

External scRNA-Seq Datasets

To complement the dataset generated in this work, prior scRNA-seq studies were included that have profiled lymphoid tissue specimens such as lymphomas, tonsils and reactive lymph nodes. For each study, the processed scRNA-seq datasets were downloaded along with the cell type annotations as provided by the authors. The cell labels were harmonized to match the 13 cell types analyzed with EcoTyper:

The scRNA-seq dataset by Roider and colleagues was obtained from heiDATA (accession code VRJUNV). (See Roider, T., et al. (2020). Dissecting intratumour heterogeneity of nodal B-cell lymphomas at the transcriptional, genetic and drug-response levels. Nat Cell Biol 22, 896-906; the disclosure of which is hereby incorporated by reference in its entirety.) The dataset includes DLBCL, transformed FL (tFL), FL and reactive lymph node tissue specimen. Myeloid cells were labeled as “Monocytes and Macrophages”, TH as “T cells CD4”, TTOX as “T cells CD8”, TREG as “Tregs” and TFH as “T cells follicular helper”. B cells labeled as “Healthy B” in tumor samples, or B cells profiled from reactive lymph nodes were assigned as “normal”, while remaining tumor B cells were assigned as “tumor”.

The follicular lymphoma dataset of Andor and colleagues was kindly shared by the authors along with the cell annotation. (See Andor, N., et al. (2019). Single-cell RNA-Seq of follicular lymphoma reveals malignant B-cell types and coexpression of T-cell immune checkpoints. Blood 133, 1119-1129; the disclosure of which is hereby incorporated by reference in its entirety.) Cells assigned to “CD14 monocytes” were labeled as “Monocytes and Macrophages”, all CD4 populations were labeled as “T cells CD4” except for cells labeled as “CD4 Regulatory T” which were assigned to “Tregs”, CD8 T cell populations were labeled as “T cells CD8” and “CD56 NK” populations as “NK cells”. Both normal and tumor B cells were included, annotated as “B cells”.

The scRNA-seq dataset from Zhang and colleagues, which consists of two samples from two FL cases, one with primary FL and progressed FL, and one with primary FL and transformed FL, were downloaded from Zenodo along with the author's cell annotation re-labeled to match the nomenclature used here. (See Zhang, A. W., et al. (2019). Probabilistic cell-type assignment of single-cell RNA-seq for tumor microenvironment profiling. Nat Methods 16, 1007-1015; the disclosure of which is hereby incorporated by reference in its entirety.)

The scRNA-seq dataset from Aoki and colleagues was kindly shared by the authors along with corresponding cell annotation. (See Aoki, T., et al. (2020). Single-Cell Transcriptome Analysis Reveals Disease-Defining T-cell Subsets in the Tumor Microenvironment of Classic Hodgkin Lymphoma. Cancer Discov 10, 406-421; the disclosure of which is hereby incorporated by reference in its entirety.) The reactive lymph node samples were included in the present study. The major lineages defined by the authors were used (B cells, Tregs, CD4 and CD8 T cells) and re-labeled to match the nomenclature used in this paper.

The tonsil dataset generated by King and colleagues was obtained from ArrayExpress (accession number MTAB-8999). (See King, H. W., et al. (2020). Antibody repertoire and gene expression dynamics of diverse human B cell states during affinity maturation. bioRxiv; the disclosure of which is hereby incorporated by reference in its entirety.) Although this dataset contained follicular dendritic cells which are of stromal origin, those were too few (<20) and where therefore not included in the analysis.

To interrogate cell types profiled by EcoTyper but not detected in the lymphoid scRNA-seq datasets, such as fibroblasts and endothelial cells, scRNA-seq datasets from solid tumors processed as described elsewhere were included.

Imputation of Cell-Type Specific Gene Expression with CIBERSORTx

To obtain the gene expression profiles of immune and stromal components of DLBCL samples, CIBERSORTx, a tool for digital cytometry and expression purification, was used. (Newman et al., 2019; cited above.)

### Estimation of Cell Type Abundance

The first step of gene expression purification is imputation of cell proportions across samples. To interrogate the major cell populations in DLBCL tumors, two previously validated signature matrices were applied: LM22, a signature matrix consisting of 22 human immune subsets; and TR4, a signature matrix consisting of 4 major populations (epithelial, endothelial, immune and fibroblasts). (See Newman et al. 2019; and Newman et al. 2015; cited above.) As LM22 is derived from Affymetrix microarray data, and the discovery cohort was profiled by RNA-seq, we applied the B-mode batch correction setting to overcome cross-platform variation when running CIBERSORTx. No batch correction step was done when applying CIBERSORTx and the TR4 signature to deconvolve tumor samples, as both input files were profiled by RNA-seq. The 22 subsets in LM22 were pooled into 11 major populations: B cells, plasma cells, CD4 and CD8 T cells, regulatory T cells, follicular helper T cells, NK cells, monocytes and macrophages, dendritic cells, neutrophils and mast cells. Eosinophils and epithelial cells were excluded from further downstream analysis. The 11 immune populations were normalized to the immune fraction inferred by TR4, so that the total fraction of the 13 cell types summed to 100%.

To validate the deconvolution performance of CIBERSORTx on multiple cell types in lymphoid tissues, artificial gene expression profiles were created using single-cell transcriptomes obtained from four scRNA-seq tumor atlases from lymphoid tissues. For each scRNA-seq dataset, defined fractions of cell types that were present were simulated in at least 200 cells. Cell fractions were sampled from a gaussian distribution based on the cell fractions imputed by CIBERSORTx applied to the discovery cohort. Negative fractions were set to 0 and the final fractions were re-normalized to sum to 1 across all 8 cell types. Using these cell fractions, 1,000 cells per dataset with were sampled replacement, summed their transcriptomes in non-log linear space into a pseudo-bulk mixture, and normalized the resulting pseudo-bulk mixture to TPM. In total, 100 pseudo-bulk mixtures were created per dataset. Finally, CIBERSORTx was applied to the mixtures with no batch correction, and the Pearson correlations of the imputed versus the ground truth cell proportions.

### Cell-Type Specific Gene Expression Imputation

Once the cell fractions for the 13 cell types are obtained, the next step in CIBERSORTx is gene expression purification. The cell fraction was provided as input to the high-resolution gene expression purification module of CIBERSORTx, along with the gene expression matrix of the discovery cohort filtered on protein-coding genes (GENCODE v24). Default parameters were used for this step.

### Implementation of EcoTyper in DLBCL

Discovery of DLBCL Cell States with Ecotyper

EcoTyper was applied to identify clusters for each cell-type specific transcriptome generated in the “Cell-type specific gene expression imputation” step. EcoTyper uses a variant of non-negative matrix factorization (NMF) to identify transcriptional cell states in purified gene expression profiles. As part of the clustering procedure, EcoTyper calculates the cophenetic coefficient for a range of cluster numbers, which helps determine the most stable number of cell state for each cell type. Following this approach, we selected the number cluster closest to a cophenetic coefficient of 0.99, a threshold that was well aligned with the elbow of the curve across all cell types, and was therefore a better fit than the default threshold of 0.95. In total, 72 cell states were defined across 13 cell types. EcoTyper applies two filters to filter out low-quality cell states. The first filter removes cell states with very few marker genes (less than 10 genes). The second filter calculates a posneg ratio filter, which removes cell states with low levels of expression and most likely to represent low-quality cell states (Luca/Steen et al., submitted). As a result, 28 cell states were filtered out, resulting in a total of 44 cell states that were used for all downstream analyses.

### Cell State Assignments of DLBCL Samples

The cell state output of EcoTyper is represented in two ways: (1) samples are represented as a mixture of cell states; (2) samples are assigned to discrete cell state, where the most dominant cell state in a given sample is assigned. In the latter, samples that are assigned to cell states filtered in the quality control step described above are excluded from the analysis.

### Recovery of DLBCL Cell States

EcoTyper provides a framework for classifying external datasets to the cell states defined in “Discovery of DLBCL cell states with Ecotyper”. This framework can be applied to independent patient cohort profiled by RNA-seq or microarray, as well as single cells profiled by scRNA-seq. EcoTyper leverages the proprieties of non-negative matrix factorization (NMF) to apply the learnt model in the discovery cohort to external datasets. Starting from a gene expression matrix, the cell state recovery framework results in a mixture coefficient matrix where each state is represented as a weight. This is done by applying the cell type-specific base matrix defined in the discovery cohort.

Using the approach for reference-guided approach to map single-cell transcriptomes to EcoTyper states, the recovery rate was compared across the various tissues types profiled by scRNa-seq. Cell types were included with full representation across tissues and at least 200 cells in each scRNA-seq dataset. The recovery of cell states was calculated across normal lymphoid tissues such as tonsils and reactive lymph nodes, tumor lymphoid tissues such as follicular lymphoma and DLBCL, and solid tumor tissues. We then compared the recovery rate for all cell types (B cells, CD4 T cells, CD8 T cells and Tregs) using a two-sided Wilcoxon's t-test.

Identification of Cellular Communities with EcoTyper

As part of the framework, Ecotyper identifies communities of the cell states defined across cell types, representing multicellular ecosystems. This is done by leveraging the Jaccard index to calculate the overlap between pairs of cell states. Starting from the 44 DLBCL cell states discovered by EcoTyper, a matrix of Jaccard indices was obtained of dimensions 44 rows×44 columns. When generating the matrix, a hypergeometric test is run for each pair of cell state, testing the null hypothesis that two cell states have no overlap, and setting the Jaccard index to 0 when non-significant. Next, hierarchical clustering is applied to the Jaccard index matrix. The optimal number of clusters is then determined by silhouette analysis. Finally, using Ecotyper, the resulting cellular communities identified in the discovery cohort can next be interrogated in external datasets.

Using this approach, cellular communities were defined specific to the ABC and GCB subtypes of DLBCL. The cellular community identification framework was applied to ABC and GCB cases separately in the discovery cohort, resulting in 4 ABC communities, and 3 GCB communities. The 7 communities were next interrogated in the 3 DLBCL validation cohorts.

Cellular communities agnostic of cell-of-origin subtypes were also defined, using all DLBCL samples in the discovery cohort. The silhouette analysis yielded 8 clusters as the optimal number. However, the two halves of the largest cluster showed clear overlap with two other clusters, and was therefore split into two clusters, resulting in 9 final clusters. These clusters constituted the DLBCL cellular communities which we termed lymphoma ecotypes (LEs).

### Selection of Cell-State Specific Marker Genes

While CIBERSORTx imputes gene expression for each cell type, it only imputes a limited number of genes per cell type. (See Newman et al. 2019; cited above.) To extend the genes expressed by a given cell state and to assess the robustness of gene expression, we leveraged the transcriptome of single cells assigned to cell states using the framework described in “Recovery of DLBCL cell states”. For each scRNA-seq dataset, a final score was calculated to prioritize marker genes from scRNA-seq data. To ensure the genes to be lymphoid specific, for cell types with representation in lymphoid datasets (B cells, Plasma cells, T cells CD8, T cells CD4, Tfh, Tregs, NK.cells, Monocytes and Macrophages), we calculated the score using the lymphoid datasets only. For the remaining cell types (fibroblast, endothelial cells, mast cells, neutrophils), we calculated the score based on the solid tumor profiled by scRNA-seq. As dendritic cells were represented in just one lymphoid dataset, both solid tumors and the tonsil scRNA-seq dataset by King and colleagues were used to calculate the top marker gene score for that cell type.

### Survival Analyses

Overall survival analysis of continuous cell state and lymphoma ecotype abundance was done using Cox proportional hazard model. Briefly, EcoTyper provides a continuous abundance value for each cell state and lymphoma ecotype. This value was provided as explanatory variable to the Cox model. For multivariate analyzes, cell-of-origin or LymphGen classes were included as covariate. Kaplan-Meier plots were used to estimate the overall survival (OS) and progression free survival (PFS) of discrete variables, such as cell state assignments. Significance was assessed by log-rank p-value. As the Chapuy et al. validation cohort had shorter follow-up time than the other DLBCL patient cohorts, all four cohorts were censored at 10 years of follow-up.

### Cell Type Abundances According to Lymphoma Ecotypes

CIBERSORTx fractions mode was run on all three validation DLBCL patient cohorts (from Chapuy et al., Ennishi et al. and Reddy et al.). The same parameters were used as described in the section Cell fraction imputation, except for the cohort by Chapuy et al. which was profiled on Affymetrix microarrays, and therefore no batch correction scheme was used when applied the LM22 signature matrix to that cohort. Next, the average cell fractions were calculated for each of the 13 cell types across the samples assigned to the nine lymphoma ecotypes for each patient cohort. Finally, the mean of the average fractions was calculated of the 4 patient cohorts.

Identifying Tumor Cells in scRNA-Seq Using Copy Number and Clonotype Status

InferCNV (v3.11), an R package to identify large-scale chromosomal copy number variations in scRNA-seq data was applied to detect which cells and cell states show evidence of copy number changes. (See Tickle T, et al. (2019). inferCNV of the Trinity CTAT Project. (Klarman Cell Observatory, Broad Institute of MIT and Harvard, Cambridge, Mass., USA.); the disclosure of which is hereby incorporated by reference in its entirety.) InferCNV requires a normal reference to normalize the malignant cells against. For the two DLBCL samples profiled by scVDJ-seq, BCR clonotype information was leveraged to identify tumor and normal cells, selecting cells with non-dominant clonotype as normal reference. In addition, as no clonotype information was available for all single cells, the variance across genes was calculated for each cell, with the hypothesis that cells showing less variation in their gene expression profile exhibit fewer or no copy number changes, and can therefore be classified as normal cells. Cells were classified into high variance and low variance using a Gaussian mixture model by applying the Mclust( ) function from the mclust R package (v5.4.5) with 10 mixture components (parameter G=1:10) and univariate data (parameter model=“V”). The cells with lowest variance using this classification scheme were assigned as normal cell. As BCR clonotype information was not available for the DLBCL samples profiled by Roider and colleagues, the reactive lymph nodes from the same dataset were used as normal reference when inferring copy number status.

### Cell State Annotation

### Gene Set Enrichment Analyses

For gene set enrichment analyses, pre-ranked Gene Set Enrichment Analysis (GSEA) was applied using the fgsea package with 10,000 permutations. (See Korotkevich, G., Sukhov, V., and Sergushichev, A. (2019). Fast gene set enrichment analysis. bioRxiv, 060012; the disclosure of which is hereby incorporated by reference in its entirety.) For gene set enrichment of B cell states in known B cell development subsets, we first obtained the average log₂ TPM of the different B cell subsets defined by Holmes and colleagues were obtained. (See Holmes, A. B., et al. (2020). Single-cell analysis of germinal-center B cells informs on lymphoma cell of origin and outcome. J Exp Med 217; the disclosure of which is hereby incorporated by reference in its entirety.) The log₂ TPM of the minor subsets was averaged to get the expression profiles of major subsets (for example, DZa and DZb were averaged to obtain a DZ profile), and next computed the fold change between each subset and the remaining subsets. The gene list we provided as input to fgsea( ) was the genes ranked based on fold change, and as input pathway, we provided the top 50 marker genes for each B cell state, selected as described in the section “Selection of cell-state specific marker genes”. For enrichment analysis of particular cell types, a pre-ranked gene list the genes was provided of purified cell populations ranked by expression, along with the top 50 marker genes for the monocyte and macrophage cell state.

### Biological Processes Up-Regulated in Tregs S2

To highlight biological processes significantly enriched in Tregs state S2, the top 100 genes assigned to Tregs S2 were selected as described in the section “Selection of cell-state specific marker genes” and provided it as input to the online tool Toppfun. (See Chen, J., et al. (2009). ToppGene Suite for gene list enrichment analysis and candidate gene prioritization. Nucleic Acids Res 37, W305-311 the disclosure of which is hereby incorporated by reference in its entirety.)

Comparison of B Cell State Distribution Between Bulk and scRNA-Seq

To compare the B cell state distribution across ABC and GCB bulk tissues, the average discrete assignments for each DLBCL cohort was calculated, and then calculated the mean across the 4 cohorts. Similarly, for the scRNA-seq samples, the cell state distribution was computed for DLBCL samples profiled by scRNA-seq and classified as GCB (n=3), and DLBCL samples classified as ABC (n=2).

### Enrichment of Normal and Tumor Cells in Cell States

To identify cell states enriched in normal cells, interrogated scRNA-seq samples were interrogated that included cells from both tumor and normal tissues. For example, the scRNA-seq dataset generated in this work included a healthy tonsil in addition to lymphoma samples. In addition, lymphoma samples that included both malignant and normal cells were also included in the enrichment analysis. For each cell state and scRNA-seq dataset, it was asked whether normal cells were significantly enriched in a given cell state using Fisher's exact test. The resulting p-values were then combined from the three datasets into a meta p-value. The same exercise was repeated for tumor cells, asking whether tumor cells were significantly enriched in a given cell state.

### Analysis of Differentiation Status of Single Cells

To identify the least and most differentiated cells in DLBCL samples by scRNA-seq, we applied CytoTRACE, a computational method that predicts the differentiation state of cells from single-cell RNA-seq data. (See Gulati, G. S., et al. (2020). Single-cell transcriptional diversity is a hallmark of developmental potential. Science 367, 405-411; the disclosure of which is hereby incorporated by reference in its entirety.) For the analyses of differentiation status, the CytoTRACE R package v0.3.3 was applied with default parameters to the scRNA-seq datasets without any prior processing other than previously described under the section “Processing of scRNA-seq datasets”. When applied to the B cells from tonsils profiled by King and colleagues, CytoTRACE was applied to all B cells, including plasmablasts, (n=43,650), and selected the phenotypes relevant for the present work (from germinal center to plasmablasts and memory B cells). Single cell transcriptomes of tonsillar B cells from King et al. were selected that had previously been assigned to cell states using reference-guided cell state annotation.

Identification of Predictive Biomarker in the REMoDL DLBCL Patient Cohort Calculate Adjusted Overall Survival z-Score for Response to RB-CHOP and R-CHOP

To identify a biomarker that predicts a greater therapeutic benefit from RB-CHOP than R-CHOP, a measure was designed that maximizes response to RB-CHOP compared to R-CHOP. Specifically, the association of each cell state abundance was first calculated, before filtering any states, with overall survival in each of the two arms, R-CHOP and RB-CHOP, using a continuous univariate cox model. The resulting z-scores from each arm were aggregated into an adjusted OS z-score by first comparing the direction (sign) of association with survival between arms. If the directions were different, the adjusted z-score was set to the z-score of the RB-CHOP arm. Otherwise, it was set to the difference between the absolute z-scores of the RB-CHOP and R-CHOP, if the difference was positive, or 0 otherwise.

### Bootstrapping of 50% of REMoDL-B Cohort

To assess the robustness of the adjusted z-score to different sample sets, sets of 25%, 50% and 75% of samples were randomly selected from the whole dataset and recalculated the adjusted z-scores. For each of the three sampling levels. The procedure was repeated 50 times with different seeds.

### Calculate Enrichment of LEs

To calculate the concordant skewness of states forming lymphoma ecotypes towards greater benefit in RB-CHOP relative to R-CHOP pre-ranked GSEA was used. Specifically, states were first ranked by the adjusted z-scores and then by z-scores in the RB-CHOP arm. Then, the pre-ranked GSEA procedure implemented in the R fgsea package was then used with parameter nperm=1,000 to test whether the states from each ecotype are enriched at the top of the ranked list.

### Leave-One-Out Cross-Validation of Kaplan-Meier Analysis for T Cell CD8 S1 Abundance

A leave-one-out cross-validation procedure was employed to assign samples in the RB-CHOP arm to T cell CD8 S1 high and respectively T cell CD8 S1 low groups. Specifically, each sample in the RB-CHOP arm was held out, and assigned it to the T cell CD8 S1 high group if the abundance of its T cell CD8 S1 in that sample was above the median of the remaining samples, and to the T cell CD8 S1 low group otherwise. For classifying samples in the R-CHOP arm, we used as cutoff the median value in across all RB-CHOP samples.

### Results:

### A Framework for Discovery of Clinically Relevant Cell States and Cellular Ecosystems in DLBCL

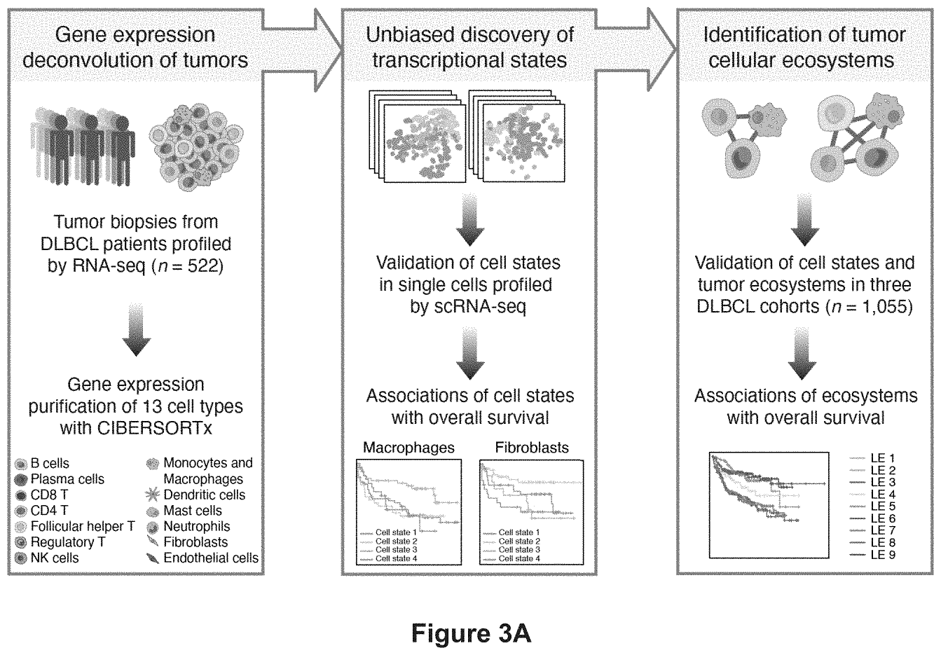

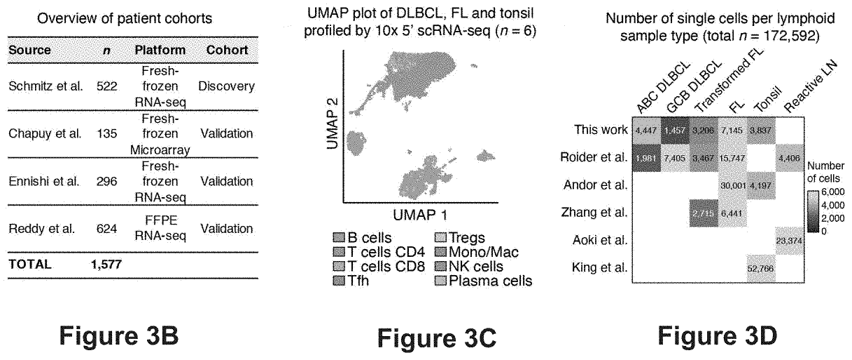

To dissect the cellular heterogeneity of DLBCL tumors, we employed EcoTyper, a computational framework for large-scale and unbiased discovery of cell states and ecosystems in tumors. EcoTyper starts by applying CIBERSORTx, an algorithm for in silico cytometry, that can reliably digitally purify the gene expression profiles of 13 cell types spanning the malignant, immune and stromal compartments of DLBCL: B cells, plasma cells, CD4 and CD8 T cells, regulatory T cells, follicular helper T cells, NK cells, monocytes and macrophages, dendritic cells, neutrophils, mast cells, endothelial cells and fibroblasts (FIG. 3A). It then clusters the cell type-specific gene expression profiles to identify distinct transcriptional programs upregulated in each cell type, referred to as transcriptional cell states. Importantly, it next identifies cell states that co-occur in the same samples, and classifies the DLBCL tumors into tumor ecosystem subtypes, termed tumor ecotypes, resulting in a landscape of the DLBCL cellular heterogeneity and its prognostic implications at a scale currently difficult to obtain experimentally.

To interrogate the cellular states and communities of DLBCL, we assembled a large number of gene expression profiles from bulk DLBCL tumors derived from patients with available clinical and genetic information, the vast majority treated with chemoimmunotherapy, resulting in a compendium of 1,577 tumors. To ensure technical robustness and extendibility across platforms, we considered various gene expression profiling platforms and tissue preservation techniques, including microarrays, and RNA-sequencing from fresh-frozen tissue and formalin-fixed paraffin-embedded (FFPE) tissues (FIG. 3B).

To validate the cell states identified by EcoTyper, we profiled by scRNA-seq 20,092 cells from normal and malignant lymphoid tissues with the 10× Genomics 5′ gene expression profiling platform (FIG. 3C). Specifically, we analyzed two DLBCL tumor specimens, one GCB and one non-GCB, three follicular lymphomas, one of which from a patient who had experienced clinical transformation, and one tonsil from a pediatric tonsillectomy. To maximize the number of cells and cell types to recover, we sorted B cells and non-B cell populations prior to sequencing library preparation. The median number of cells per sample after sequencing was 4,325 (1,970-5,645), and the median number of genes per cell was 1,581 (1,333-2,794). To further expand our single cell validation dataset, we also included samples from prior studies. This effort resulted in a pan-lymphoid atlas of 172,592 single cells spanning six studies and six lymphoid tissue types (FIG. 3D), making this, to our knowledge, the most comprehensive integration of lymphoid scRNA-seq atlases to date. To interrogate cell states in cell types that were not covered in the lymphoid datasets, such as fibroblasts, neutrophils and endothelial cells, we included a set of scRNA-seq atlases derived from solid tumors.

The EcoTyper framework, along with the extensive transcriptomic and clinical resources we assembled, set the foundation for a deep characterization of cell states present in DLBCL, as well as their clinical relevance and their co-occurrence in cellular communities.

Integrative Analysis of Purified B Cells from Bulk and Single Cell DLBCL Gene Expression Datasets

DLBCL is routinely classified into two B cell states according to cell-of-origin, activated B cell (ABC) or germinal center B cell (GCB) states. Yet, a large portion of patients (11-21%) remain unclassified, and cell-of-origin classification is currently not guiding first-line treatment. We hypothesized that the B cell states that make up DLBCL tumors, as well as their clinical phenotype, could be further refined. We applied EcoTyper to the discovery cohort consisting of 522 DLBCL tumors profiled by RNA-seq from fresh-frozen tissue, resulting in the first large-scale analysis of purified B cells from DLBCL tumors. This unique resource allowed us to address key questions related to the diversity of B cell states in DLBCL, such as their robustness across datasets, their prognostic associations, and their link to known DLBCL subtypes.

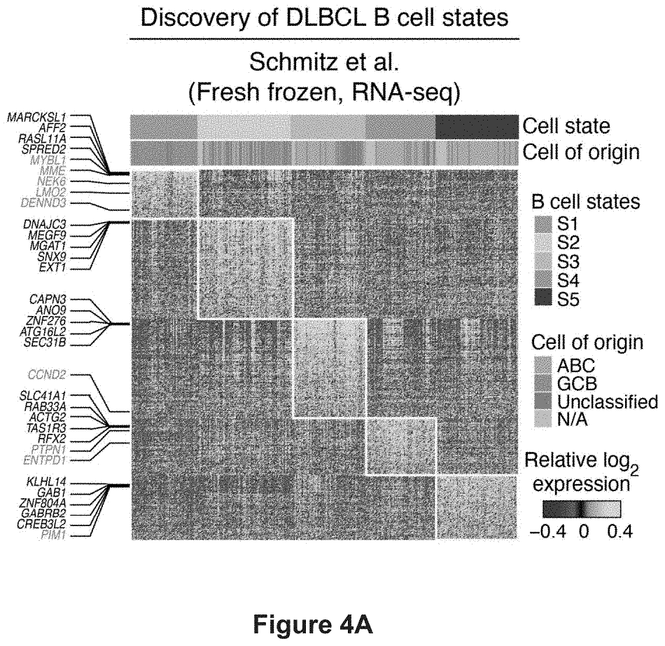

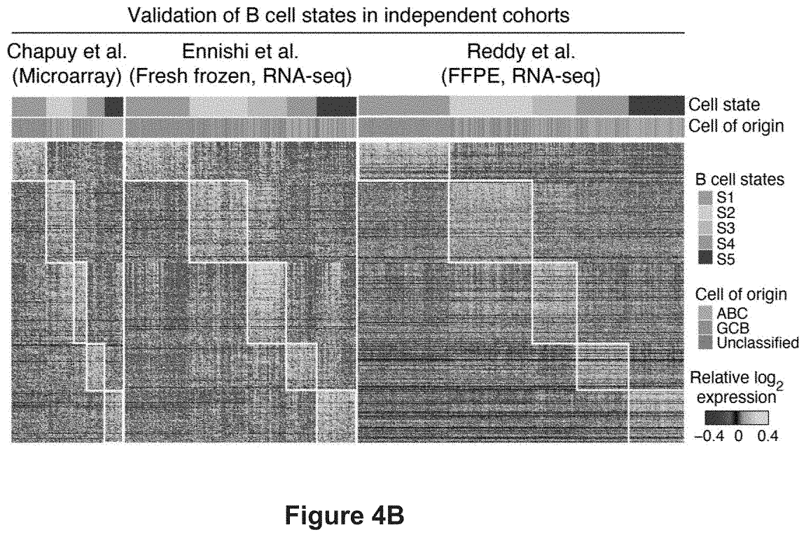

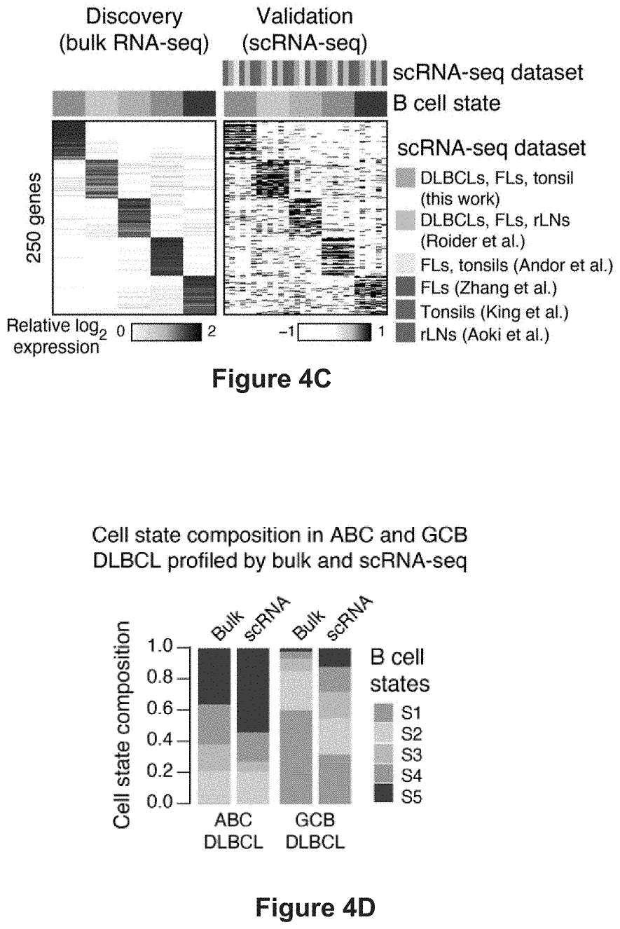

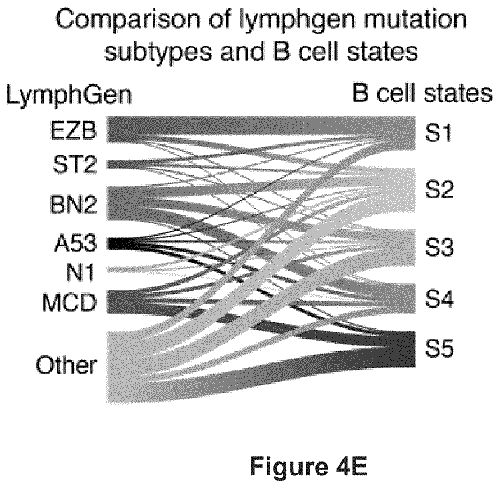

We first asked if the purified B cell transcriptomes from DLBCL tumors exhibited more granularity than the previously defined ABC and GCB DLBCL classes. Indeed, EcoTyper subdivided DLBCL B cells into five distinct cell states (FIG. 4A), suggesting that DLBCL B cell heterogeneity extends beyond the ABC and GCB dichotomy. The B cell states differed in their gene expression profiles and the distribution of cell-of-origin classes. B cell state S1 expressed genes that are typically observed in the GCB subtype of DLBCL, such as MME, LMO2, MYBL1 and BCL6. In fact, it consisted almost exclusively of DLBCL samples assigned to the GCB cell class (Fisher's exact test; P=1.2×10⁻³⁹), thereby representing a dominant germinal center B cell state. In contrast, B cell states S4 and S5 were significantly enriched for ABC DLBCL samples (Fisher's exact test; P=5.0×10⁻⁶ and P=4×10⁻¹⁶, respectively), as well as expressing known ABC DLBCL genes, such as PIM1, and PTPN1. While B cell states 51, S4 and S5 recapitulated to some extent the known cell-of-origin states of DLBCL, B cell states S2 and S3 on the other hand represented hybrids of ABC, GCB and unclassified DLBCLs, thereby revealing more granular subtypes of DLBCL.

Our DLBCL B cell atlas serves as a resource to further explore these states and their marker genes, such as cell surface proteins or key transcription factors. While B cell state S1 expresses transcription factors known to be specific to GCB DLBCL, the other cell states express lesser known markers in DLBCL. For example, ZEB2, a transcription factor involved in epithelial-mesenchymal transition in development and epithelial cancers, is highly specific for B cell state S2. While its role in lymphoma is less clear, it has been shown to be an oncogenic driver of immature T-cell acute lymphoblastic leukemia. (See Goossens, S., et al. (2017). Oncogenic ZEB2 activation drives sensitivity toward KDM1A inhibition in T-cell acute lymphoblastic leukemia. Blood 129, 981-990; the disclosure of which is hereby incorporated by reference in its entirety.) A key transcription factor of B cell state S3 is ZNF276, which codes for a protein that can be down-regulated by pomalidomide, a drug that has recently shown promising results in combination with dexamethasone in relapsed/refractory primary central nervous system lymphoma. (See Sievers, Q. L., et al. (2018). Defining the human C2H2 zinc finger degrome targeted by thalidomide analogs through CRBN. Science 362; and Tun, H. W., et al. (2018). Phase 1 study of pomalidomide and dexamethasone for relapsed/refractory primary CNS or vitreoretinal lymphoma. Blood 132, 2240-2248; the disclosures of which are hereby incorporated by reference in their entireties.) B cell state S4 shows high expression of BATF, a transcription factor that mediates class-switch recombination in B cells (Ise et al., 2011), while TCF4 is highly specific to B cell state 5. (See Ise, W., et al. (2011). The transcription factor BATF controls the global regulators of class-switch recombination in both B cells and T cells. Nat Immunol 12, 536-543; the disclosure of which is hereby incorporated by reference in its entirety.) Of note, TCF4 has recently been shown to be down-regulated by specific therapeutic targets in a pre-clinical study in ABC DLBCL. (See Jain, N., et al. (2019). Targetable genetic alterations of TCF4 (E2-2) drive immunoglobulin expression in diffuse large B cell lymphoma. Sci Transl Med 11; the disclosure of which is hereby incorporated by reference in its entirety.)

To assess the reproducibility of the five B cell states identified in the discovery cohort, we next asked if we could recover the same cell states in independent DLBCL tumors. EcoTyper provides a framework for classifying new samples into defined cell states. We applied the classifier to three DLBCL cohorts, classifying in total 1,577 DLBCL tumor transcriptomes into five B cell states. Strikingly, the cell-state specific pattern of gene expression observed in the discovery cohort was broadly recapitulated in the validation cohorts (FIG. 4B), and the cell-of-origin enrichments were highly concordant, demonstrating the reproducibility of the B cell states. Importantly, as these cohorts were derived from various gene expression profiling platforms and tissue presentation techniques, these results demonstrate that the B cell states are highly robust across tissue specimens and profiling technologies.

Having shown that B cell states are reproducible across DLBCL patient cohorts, we next asked whether they are detectable in scRNA-seq data. Using the same approach of interrogating B cell states in independent DLBCL tumors, we applied the EcoTyper classifier to B cells from two lymphoid tissues profiled by scRNA-seq, including multiple DLBCLs samples. Indeed, we could reproduce the strong concordance of marker genes in the single cells assigned to the five B cell states and their significant validation (FIG. 4C). Given these significant results across 12,000 B cells from six distinct datasets and spanning a total of 36 scRNA-seq samples, we were confident that the B cell states derived from digitally purified DLBCL tumors could be detected in scRNA-seq data and were therefore real.

Whole-exome sequencing and scRNA-seq studies have shown that tumors, including lymphomas, do not consist of a unique tumor clone, but rather may consist of multiple co-existing subclones. Similarly, we hypothesized that DLBCL tumors could comprise more than one transcriptional cell state. Indeed, when we compared the distribution of B cell states in DLBCL tumors classified as ABC or GCB DLBCL, we observed that the ABC and GCB samples did not consist of one unique cell state, but rather of a mixture of cell states (FIG. 4D). Notably, the cell state composition in ABC and GCB was highly conserved across purified DLBCL bulk and scRNA-seq samples with known cell-of-origin labels (FIG. 4D). This observation suggests that a DLBCL tumor cannot necessarily be classified into a single class according to cell-of-origin, but rather, that the cell state composition of a tumor is a more reliable measure of B cell heterogeneity. Importantly, while genetic subclones are private to a specific tumor and patient, these results show that the EcoTyper cell states are ubiquitous and generalize across patients.

Having shown that the B cell states defined by EcoTyper extend across patient cohorts and single cell atlases, we next investigated if they were linked to specific mutation profiles of DLBCL tumors. While early evidence of heterogeneity in DLBCL has been linked to differences in gene expression, more recent studies have defined new subtypes of DLBCL based on distinct mutational profiles. (See Alizadeh, A. A., et al. (2000). Distinct types of diffuse large B-cell lymphoma identified by gene expression profiling. Nature 403, 503-511; Chapuy, B., et al. (2018). Molecular subtypes of diffuse large B cell lymphoma are associated with distinct pathogenic mechanisms and outcomes. Nat Med 24, 679-690; Schmitz, R., et al. (2018). Genetics and Pathogenesis of Diffuse Large B-Cell Lymphoma. N Engl J Med 378, 1396-1407; and Wright, G. W., et al. (2020). A Probabilistic Classification Tool for Genetic Subtypes of Diffuse Large B Cell Lymphoma with Therapeutic Implications. Cancer Cell 37, 551-568 e514; the disclosures of which are hereby incorporated by reference in their entireties.) These studies have expanded the number of DLBCL classes from two classes to five or more, and we wondered if the EcoTyper B cell states were a reflection of variation across mutational classes. Nevertheless, when we compared the B cell states with mutational subtypes, although some cell states did show enrichment of certain subtypes and their key mutations, and this enrichment was consistent across cohorts, there was not a one-to-one relationship between mutation profiles and transcriptional B cell states (FIG. 4E). Furthermore, a large proportion of unclassified tumors based on mutational profiles was represented across all five B cell states. These results support that the B cell states stratify DLBLC into novel subtypes, thereby revealing new heterogeneity in DLBCL beyond previously defined classes.

B Cell States are Heterogeneous in their Prognostic Association, Spatial Distribution, and Developmental Stage

While others have previously shown the prognostic relevance of B cell subsets in DLBCL, these were defined in bulk tumors or from B cells purified from normal tissues. (See e.g., Holmes, A. B., et al. (2020). Single-cell analysis of germinal-center B cells informs on lymphoma cell of origin and outcome. J Exp Med 217; the disclosure of which is hereby incorporated by reference in its entirety). In contrast, we purified B cells specifically from DLBCL tumors, and therefore hypothesized that the EcoTyper-derived B cell states could refine current prognostic associations in DLBCL. While B cell state 51, the pure GCB cell state, was as expected significantly associated with longer overall survival (P=4.8×10⁻⁵), it was in fact the most favorable cell state, confirming the more indolent property of GCB DLBCL tumors. Likewise, B cell state S5, which was most significantly enriched for ABC DLBCL, was associated with shortest overall survival (P=4.9×10⁻⁷). Interestingly, this association was maintained in a multivariate analysis adjusting for cell-of-origin (P=0.03) and mutational subtypes respectively (P=0.02). In contrast, B cell state 4, also significantly enriched for ABC tumors, was not significantly associated with overall survival. Similarly, B cell state S3, a cell-of-origin hybrid state, was not significantly associated with outcome. B cell state S2 on the other hand, also a hybrid state, was significantly associated with longer overall survival (P=0.0002), also after adjusting for cell-of-origin (P=0.0002) and mutational subtypes (P=0.02), thus representing a novel B cell state with prognostic significance independent of molecular subtypes.

While the B cell states were identified in B cells purified from tumor samples, a tumor may consist a both normal and tumor cells. As the scRNA-seq datasets used for validation included both malignant and normal B cells, we therefore asked if the B cell states were more enriched for normal B cells. While all five cell states showed representation in tumor and normal samples, B cell state S3 was significantly over-represented in non-malignant B cells, a finding consistent across three independent scRNA-seq datasets (Fisher's exact test meta-P=1.6×10⁻⁵⁸).

To further characterize the novel DLBCL B cell states S2 and S3, we interrogated a normal lymph node profiled by spatial transcriptomics (10× Visium), allowing us to study the spatial localization of cell states across the tissue. While the cell state distribution in the lymph node profiled by spatial transcriptomics was comparable to reactive lymph nodes profiled by scRNA-seq, S4 and S5 were practically non-existent in the spatial transcriptomics dataset. We observed that the remaining states S1, S2 and S3, exhibited clear distinct spatial distribution. As expected, B cell state S1, the GCB cell state, was confined to the follicles. In contrast, S2 and S3 seemed to exhibit a gradient going from inside to outside the follicles.

A pattern of migration within the lymph node could potentially reflect various states of cell differentiation. Based on the variation in spatial distribution, we therefore wondered if the cell states in the normal lymph node represented distinct differentiation states of B cells. Indeed, when we applied scRNA-seq data from non-neoplastic lymph nodes to CytoTRACE, an algorithm that predicts differentiation status of cells based on a measure of transcriptional diversity, we confirmed that S1 was least differentiated, while S3 was most differentiated, supporting a migratory trajectory moving from the follicles to outside the follicles. Notably, this differentiation ordering was conserved in tumor samples.

Prior studies have assigned ABC DLBCL tumors to differentiated B cell states that are maturing to become plasmablasts, the final stage of B cell differentiation. Having determined the differentiation status of B cell states S1, S2 and S3 in non-neoplastic lymph nodes and tumors, we next asked if the ABC-like states S4 and S5 showed indeed a more differentiated state. Surprisingly, these two cell states were less or equally less differentiated compared to the GC-like B cell state 1, both in normal and tumor samples, suggesting that B cell states S4 and S5 may arise from a progenitor cell prior to the germinal center reaction. Notably, S5 was most highly enriched for a pre-GC B cell state described by King and colleagues in tonsil, supporting that this ABC-like state may arise from an earlier differentiation B cell state than previously thought.

In summary, we describe five B cell states of DLBCL, one of which is a pure GCB state and favorable, while two are dominantly ABC, one of them being adverse and potentially arising from a pre-GC B cell state. B cell state S3 is a more normal and differentiated state, and B cell state S2 represents a novel prognostic B cell state independent of cell-of-origin, and marking patients of superior outcome.

### The Prognostic Landscape of the DLBCL Tumor Microenvironment

Early gene expression studies identified inflammatory and stromal gene signatures as prognostic in DLBCL, but these studies were performed in bulk DLBCL tumors, and did not decouple the TME from the B cell compartment. (See Lenz, G., et al. (2008). Stromal gene signatures in large-B-cell lymphomas. N Engl J Med 359, 2313-2323; and Monti, S., et al. (2005). Molecular profiling of diffuse large B-cell lymphoma identifies robust subtypes including one characterized by host inflammatory response. Blood 105, 1851-1861; the disclosures of which are hereby incorporated by reference in their entireties.) Importantly, this approach cannot pinpoint the specific cell type or cell state with prognostic significance. In contrast, having purified the transcriptome of 12 cell types of the DLBCL TME, we could now systematically catalogue the diversity and clinical relevance of the TME cell states.

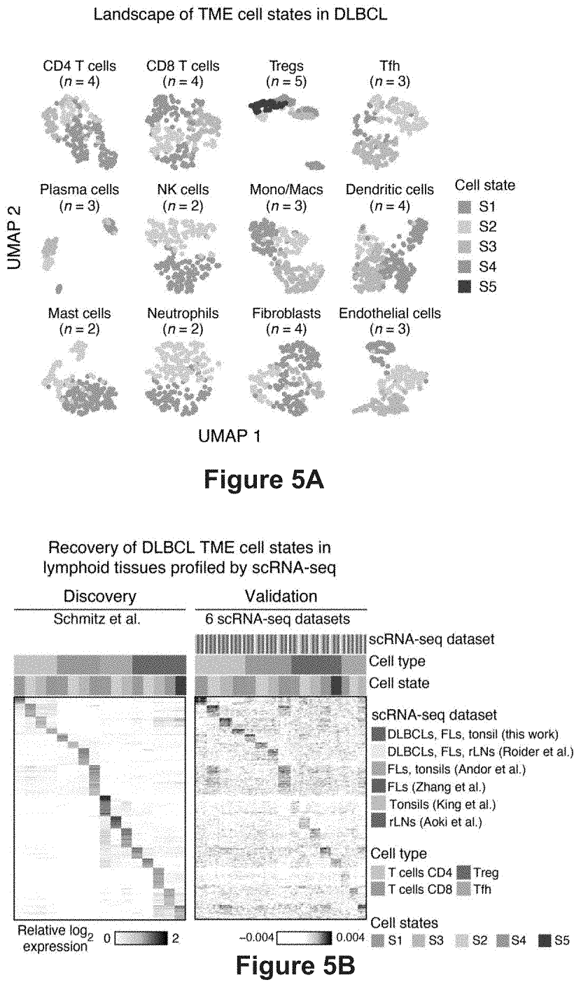

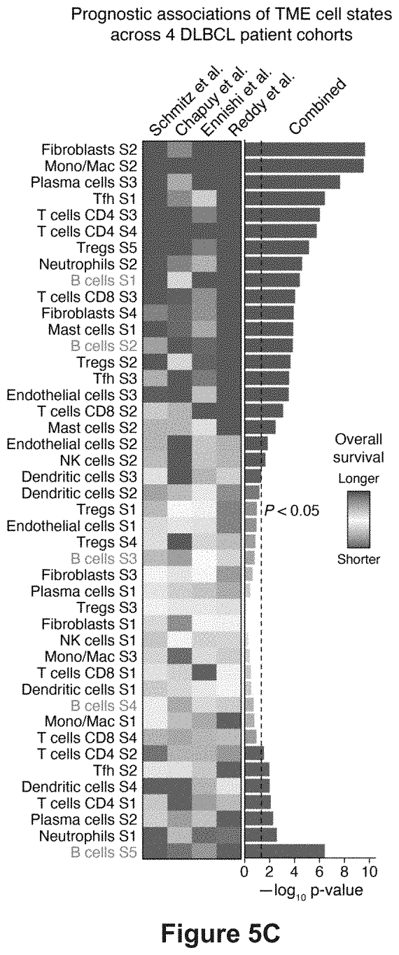

Analogous to how we defined five cell states for B cells, we defined cell states of 12 cell types of the DLBCL TME, including lymphoid, myeloid and stromal populations (FIG. 5A). The number of states ranged from 2 to 5 depending on the cell type, resulting in an atlas of 39 distinct transcriptional states across 12 major cell types, which were detectable in all four DLBCL patient cohorts.

Similar to B cells, we interrogated lymphoid scRNA-seq atlases for the various TME cell states (FIG. 5B). Using this approach, 25 out of 30 cell states (83%) of TME cell types profiled in lymphoid scRNA-seq could be significantly recovered. Importantly, the expression of top transcription factors and cell surface proteins was highly concordant across scRNA-seq. To recover cell types typically lost due to dissociation distortions in lymphoid cell suspensions, we considered atlases from solid tumors where non-malignant cell populations of the TME had been profiled. This enabled us to recover 11 additional cell states, resulting in a total recovery rate of 91% (41/44) including B cell states. Of note, when we compared the recovery rate of cell types that overlapped between solid tumors scRNA-seq atlases and lymphoid atlases, the rate was significantly higher in lymphoid datasets (P=0.02; two-sided Wilcoxon rank sum test), supporting the evidence that these cell types are more specific to lymphoid tissues. Similarly, the DLBCL TME cell states showed higher recovery rate in tumor samples compared to normal tonsils (P=0.01).

Having decoupled the TME cell states from the malignant compartment, and given the extensive diversity in cell states, we now had the opportunity to examine the survival associations of TME cell states, resulting in a unique atlas of the prognostic TME of DLBCL (FIG. 5C). Remarkably, the survival associations were highly concordant and significantly correlated between the discovery and validation cohorts, with the majority of the cell states significantly prognostic across all 4 cohorts, and two thirds (62%) significant in a multivariate analysis including cell-of-origin. Importantly, for several cell types, we could detect an adverse cell state and a favorable counterpart, underscoring that heterogeneity is not restricted to the level of cell types, but rather at the level of cell states. Unexpectedly, while ABC-like B cell state S5 was the state most significantly associated with shorter survival, the top eight most favorable cell states belonged to the TME. Notably, seven of these TME cell states maintained their prognostic effect after adjusting for cell-of-origin, demonstrating that the TME plays a key role in the clinical phenotype of DLBCL.

### EcoTyper Reveals Cell-of-Origin Specific TME Cell States and Ecosystems

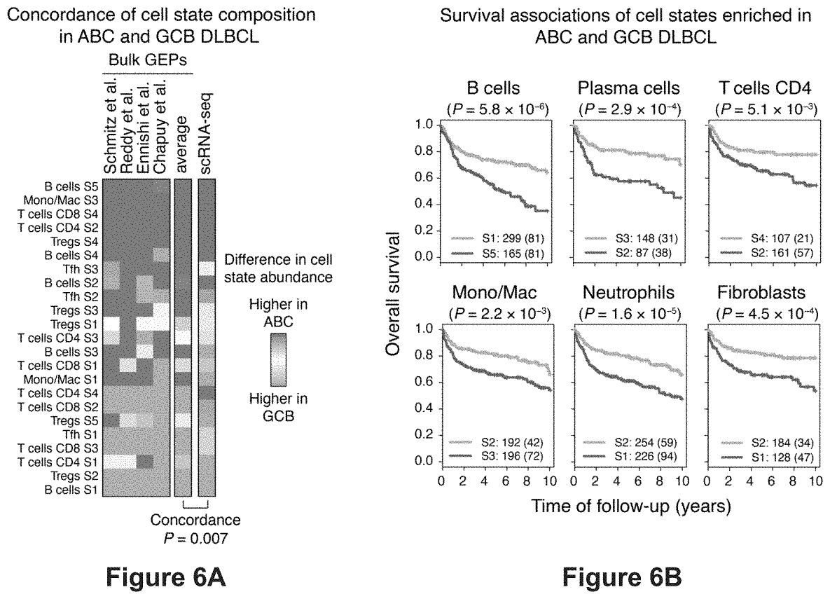

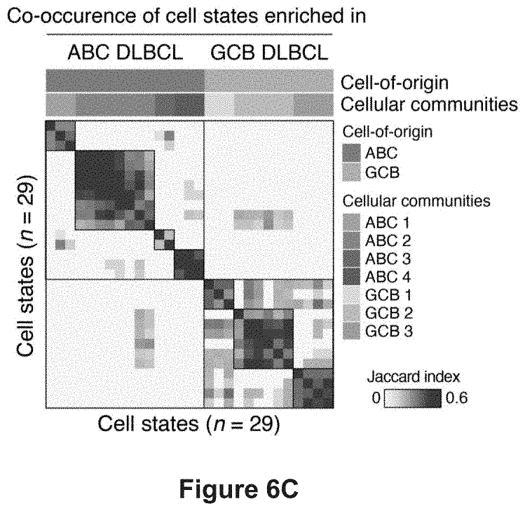

While GCB and non-GCB tumors have been profiled by scRNA-seq, cell-of-origin specific differences related to TME cell states have not been described. Having revealed the landscape of the TME cell states in DLBCL and their prognostic significance, we now had the opportunity to examine TME cell state composition in the context of cell-of-origin. We identified cell states significantly enriched in digitally purified cell types from ABC and GCB DLBCL tumors, and asked if we could detect the same enrichment in samples profiled by scRNA-seq with known cell-of-origin labels. Indeed, there was a significant concordance in ABC and GCB enrichments when comparing to the EcoTyper-derived cell states (Spearman correlation P=0.01, FIG. 6A). Importantly, both the purified bulk and scRNA-seq DLBCL tumors were classified into EcoTyper-derived TME cell states blinded to cell-of-origin classes, further emphasizing the robustness of TME cell states and their link to cell-of-origin in DLBCL. Furthermore, the survival curves of cell states for each cell type mirrored the survival associations of cell-of-origin in bulk samples, where GCB TME cell states were associated with longer overall survival time compared to ABC TME cell states (FIG. 6B).

This analysis provided a unique opportunity to contrast the biology of TME cell states specific to ABC and GCB. For example, The ABC CD4 T cell state showed higher expression of co-stimulatory and co-inhibitory molecules such as LAG3, HAVCR2 (TIM3), and CTLA4, while the CD4 T cell state most enriched for GCB DLBCL was nearly depleted for these molecules, but in contrast showed high expression of TIGIT and ICOS, highlighting fundamental differences in the immunology of the two cell-of-origin subtypes.

We noted that some cell state showed low correlations in their abundance with other cell states within the same cell-of-origin class, indicating a more complex structure than a simple dichotomy of the ABC/GCB TME (FIG. 6C). Indeed, hierarchical clustering revealed four distinct subgrouping of cell states within the ABC class, and three subgroupings within the GCB class, reflecting distinct TME substructures, or distinct tumor ecosystem subtypes, linked to cell-of-origin. Importantly, these tumor ecosystem subtypes showed differences in their overall survival associations within the cell-of-origin classes, representing clinically relevant cell state communities exhibiting biological differences. Together, these results suggest that there is not a unique TME pertaining to ABC or to GCB, but rather that several cell states communities make up biologically and clinically distinct tumor ecosystem subtypes in DLBCL tumors.

DLBCL Cell States Form Distinct Tumor Ecosystems that are Prognostic Independently of Cell-of-Origin and Mutational Subtypes

The previous analysis demonstrated that there is more complex structure in the DLBCL TME than previously appreciated, and this structure is not restricted to cell-of-origin subtypes. To extend these results, we therefore asked if we could define communities of cell states present in DLBCL tumors agnostic to cell-of-origin.

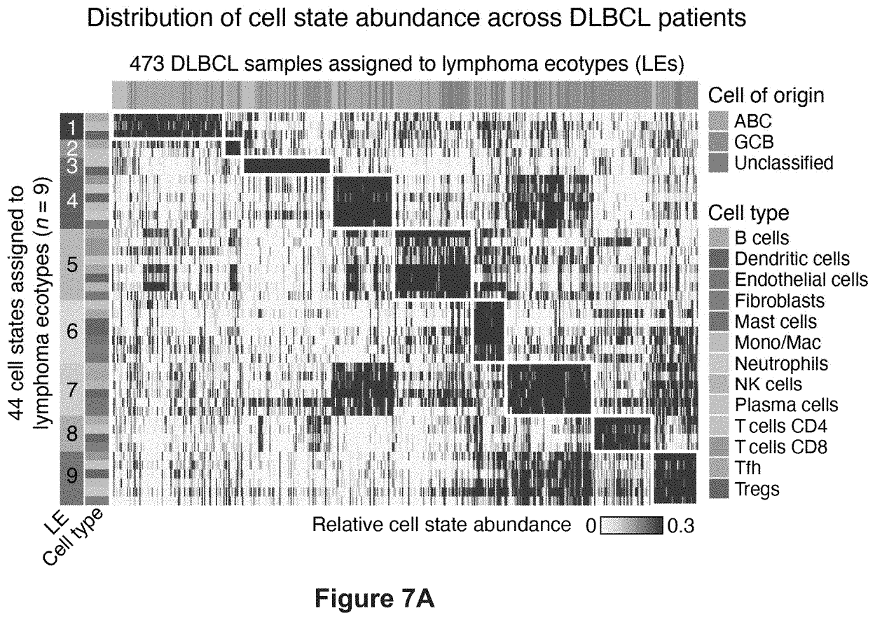

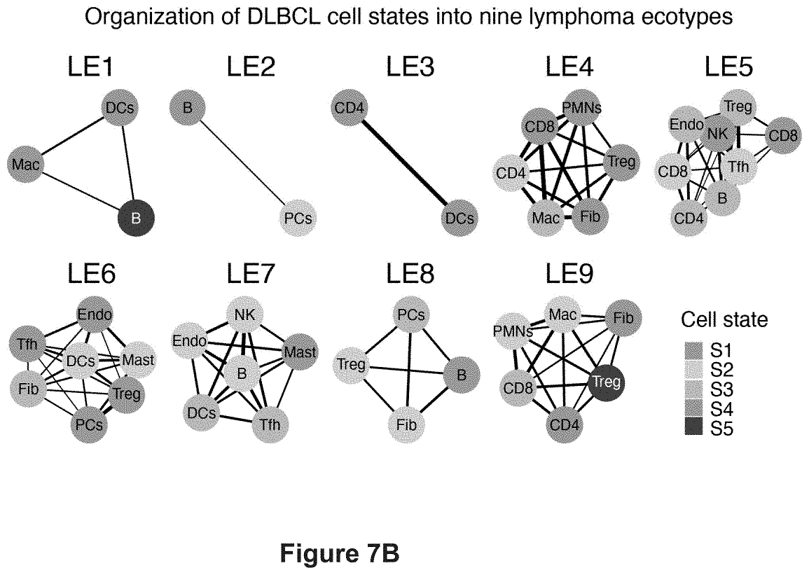

Analogous to how we defined ABC and GCB DLBCL TME subgroups, EcoTyper identifies cell states that overlap across tumor samples, and group them into cellular communities, termed tumor ecotypes. We applied this approach to the discovery cohort, and defined nine distinct DLBCL tumor ecotypes, which varied in their number of component cell states (FIGS. 7A-7B). TE1 and TE2 for example consisted of three and two cell states respectively, each one with a B cell state, representing potentially more B cell dominant tumor ecotypes, while TE4, TE5, TE6, TE7 and TE9 consisted of six cell states cell states or more, reflecting a more diverse and richer tumor microenvironment.

Similar to how we could classify independent datasets into cell states of the DLBCL TME, EcoTyper provides a framework for classification into tumor ecotypes. To determine the generalizability of the DLBCL tumor ecotypes, we therefore applied the classifier to the three DLBCL validation cohorts. The vast majority of samples could be significantly assigned to a tumor ecotype (92% in total, range 91-93%), and the distribution of cell state abundance across the four studies was strikingly similar, exhibiting clear co-associations in each individual dataset.

Having shown that the ecotypes were robust across DLBCL cohorts, we next investigated their clinical relevance. Remarkably, the prognostic associations of the tumor ecotypes were highly conserved across datasets (FIG. 7C), and reflected the cell-state specific continuous survival associations. When we combined the survival associations with overall survival into a weighted meta p-value, eight out of the nine ecotypes were significantly prognostic. TE1, TE2, TE3 and TE4 were significantly associated with shorter overall survival time, while TE6, TE7, TE8 and TE9 were significantly associated with longer overall survival time. Importantly, more than half (5/9) were prognostic independently of cell-of-origin or Lymphgen mutational subtypes. Of note, the strong survival associations of the tumor ecotypes underscore the power of considering several cell states when examining survival associations. For example, while the ABC-DLBCL enriched B cell state S4 was not significantly associated with adverse survival alone, together with plasma cell S2 they constitute a highly adverse tumor ecotype. Likewise, TE8, the tumor ecotype that comprises B cell state S1 which the most favorable B cell state, is superseded by the more favorable and TME-rich tumor ecotype TE9. Strikingly, while TE5 was the only tumor ecotype not significantly associated with overall survival, all of the cell states we had previously shown to be enriched for normal cells in scRNA-seq co-associated into that specific tumor ecotype. Importantly, these cell states were grouped together into a single tumor ecotype without prior knowledge of normal enrichment, as our discovery cohort did not include normal bulk samples.

Having defined distinct prognostic tumor ecosystems in DLBCL, we next explored their associations with cellular composition and known molecular subtypes. The cell type abundance and subtype enrichments were highly concordant across the discovery and validation cohorts As suggested by their lower number of component cell states, the adverse tumor ecotypes TE1 and TE2 were indeed more enriched for B cell and plasma cells. In contrast, the more favorable tumor ecotypes, except from TE8, showed a higher abundance of stromal and non-B cell immune populations. TE8, which showed a more B cell enriched cellular composition compared to other favorable TEs, consists almost exclusively of GCB DLBCL samples. As both ABC and GCB enriched tumor ecotypes showed higher B cell content, it could be that these ecosystems are more strongly driven by the B cell compartment, rather than the TME. Interestingly, TE9, the most favorable tumor ecotype, showed highest abundance of stromal cells such as fibroblasts and endothelial cells compared to other TEs. While it has been shown that stromal signatures are associated with favorable outcome in DLBCL, TE9 was only modestly enriched for GCB and unclassified DLBCL, and did not show any significant enrichment of mutational subtypes (FIG. 7C). Likewise, TE7, a favorable tumor ecotype with a component B cell state, showed no overlap with previously defined molecular subtypes. Thus, TE7 and TE9 represent novel subtypes of DLBCL with diverse and favorable tumor microenvironments.

In summary, the cell states communities represent distinct clinically-relevant tumor ecosystems in DLBCL, that are independent of cell-of-origin and mutational subtypes. T cells CD8 S1 is a biomarker for response to Bortezomib in DLBCL

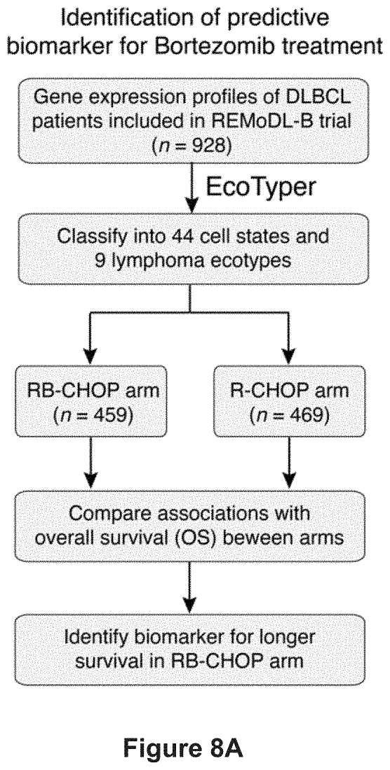

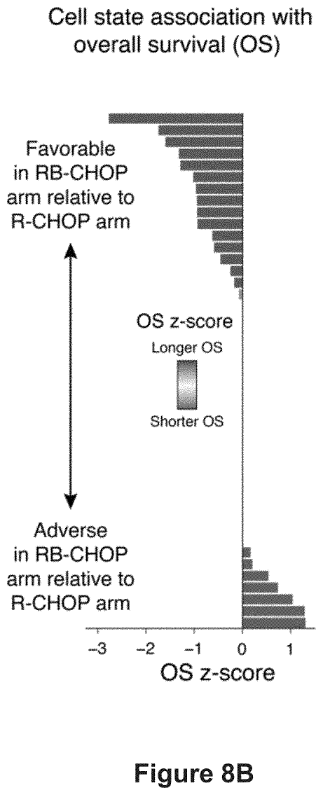

While there are clinical biomarkers for risk stratification of DLBCL patients, such as the use of Ann Arbor stage or the International Prognostic Index (IPI), these biomarkers do not guide treatment selection at diagnosis. The atlas of cell states and ecosystems defined with EcoTyper serves as a resource for identifying potential novel predictors for treatment outcome in DLBCL. To illustrate this, we applied the DLBCL EcoTyper classifier to a cohort derived from the REMoDL-B clinical trial, a clinical cohort that includes 928 DLBCL tumors analyzed by gene expression profiling prior to treatment initiation (FIG. 8A). The trial tested the standard of care in DLBCL (rituximab in combination with chemotherapy; the R-CHOP arm) against the addition of the drug bortezomib to R-CHOP (the RB-CHOP arm). (See Davies, A., et al. (2019). Gene-expression profiling of bortezomib added to standard chemoimmunotherapy for diffuse large B-cell lymphoma (REMoDL-B): an open-label, randomised, phase 3 trial. Lancet Oncol 20, 649-662; the disclosure of which is hereby incorporated by reference in its entirety.) While the results of the trial results were negative, with no significant differences in outcomes between the two treatment arms, interestingly, cell states that constitute LE5 were over-represented among the cell states most favorable in the RB-CHOP arm relative to the R-CHOP arm (FIG. 8B). Importantly, by comparing outcomes between the R-CHOP and RB-CHOP arms, we recapitulated the co-occurrence of cell states into ecotypes independently of their original definition.

Among the cell states most strongly associated with longer overall survival in the RB-CHOP arm relative to the R-CHOP arm, T cell CD8 S1 was the most significant cell state. We derived a classifier based on T cell CD8 S1 abundance that significantly stratified patients within the RB-CHOP arm (P<0.0001) in a leave-one-out cross-validation setting. Moreover, patients with high T cell CD8 S1 content showed more favorable outcome when treated with RB-CHOP than patients treated with R-CHOP (P=0.03), thus resulting in a significant outcome of the clinical trial. Importantly, this biomarker stratified patients within the ABC DLBCL subtype. Pre-clinical studies suggested that the addition of bortezomib might be more efficient in ABC-DLBCL cases. However, the results of the REMoDL-B trial did not support this hypothesis. Here, we show that we can identify the ABC DLBCL most likely to respond to bortezomib. Although bortezomib was though to target the constitutively active NF-κB pathway in B cells of ABC-DLBCL tumors, these results suggest that the efficacity of bortezomib might be instead on the surrounding T cells. T cells CD8 S1 express CXCR5, and may therefore reflect a CXCR5+CD8+ population recently described as present in follicular lymphoma and showing antitumor activity. Indeed, when we did an enrichment analysis of the CD8 T cell S1 marker genes in CXCR5 positive, CXCR5 negative, and naïve CD8 T cells, the enrichment was highest in the CXCR5 positive population.

DISCUSSION: Although the introduction of rituximab for treatment of DLBCL has dramatically improved survival, DLBCL is still uncurable for nearly half of the patients, and outcomes are poor for patients who do relapse. More recently, several therapies that harness the immune system have been approved to treat patient who have relapsed, for example CAR T cells in 2017, and others are currently being investigated. While the TME of DLBCL tumors and its potential impact on survival has previously been explored, large scale studies did not decouple the gene expression of the TME cell types from the malignant compartment, or they were limited to specific cell subsets and sets of markers. In this study, we present an unprecedented atlas of the prognostic cell states and ecosystems that constitute the DLBCL TME.

This study distinguishes itself from prior studies of the DLBCL TME on several important points. Firstly, we provide the largest study to date of purified B cell transcriptomes in DLBCL. While other groups have purified B cell populations and obtained their gene expression profiles, the studies done on DLBCL tumors were of modest size, while other studies on B cell states were restricted to normal lymphoid tissues. Here we provide a portrait of the transcriptional and prognostic diversity of B cells purified from DLBCL tumors specifically, and show how they differ in their spatial and differentiation dynamics, as well as prognostic associations. Although Holmes and colleagues derived a classifier of B cell states and applied it to DLBCL tumors, they derived the states from two non-neoplastic tissues, tonsils, not from DLBCL tumors. Here, we show that one of the B cell states is highly normal-enriched while two the B cell states are highly tumor-enriched, a finding that cannot be easily obtained when starting from normal B cells only, underscoring the strength of defining cell states from tumor samples directly.

Secondly, we derived cell states of 12 cell types of the DLBCL TME. Lenz and colleagues reported in 2008 two transcriptional signatures enriched in stromal genes, suggesting that the tumor microenvironment plays a role in survival of DLBCL. (See Lenz et al., 2008; cited above.) Here, we confirm the important prognostic role of the TME in DLBCL, and further dissect the cellular heterogeneity in DLBCL across the entire tumor microenvironment at cell-type level, allowing to decipher the specific cell type and cell states of the TME that are associated for longer and shorter overall survival time.

Finally, while prior scRNA-seq studies have described various cell states present in the normal and neoplastic microenvironment of lymphoid tissues, they have not addressed how cell states co-associate to form ecosystems and their clinical relevance. Here, we show that ABC and GCB patients exhibit distinct TMEs, and these can be further classified into tumor ecosystem subtypes. Although cell-of-origin classification may guide treatment selection for patient who relapse, it currently does not affect the choice of first-line therapy. We show that the cell-of-origin classification extends beyond the malignant cells, and that cells of the TME exhibit distinct biological and clinical differences in relation to the ABC and GCB subtypes of DLBCL, representing potential candidates for immunotherapy stratified according to cell-of-origin subtypes. Importantly, we defined nine distinct tumor ecosystems in DLBCL beyond cell-of-origin, which show high variation in their cellular composition and enrichment for previously described molecular subtypes.

In summary, we employed a novel computational framework to digitally dissect the DLBCL cellular heterogeneity and describe an atlas of novel states for diverse cell types in these tumors. We show how cellular states form cohesive tumor ecosystems, which exhibit distinct clinical outcomes. These results expand our understanding of cellular heterogeneity in DLBCL, and may have implications for the development of novel individualized therapies, as well as potentially improving diagnostics and identifying novel biomarkers.

### Example 2: An Atlas of Clinically Distinct Cell States and Cellular Ecosystems Across Human Solid Tumors

BACKGROUND: Previous studies have revealed broad phenotypic classes in human tumors, ranging from tumors that are T cell-inflamed (“hot”) to those that are T cell-depleted (“cold”). (See Binnewies, M., et al. (2018). Understanding the tumor immune microenvironment (TIME) for effective therapy. Nature Medicine 24, 541-550; the disclosure of which is hereby incorporated by reference in its entirety.) Such classifications can inform disease characteristics, including response to ICI, but oversimplify the cell types and cellular states of the tumor microenvironment (TME). In recent years, single-cell genomics, spatial transcriptomics, and multiplexed imaging have emerged as powerful technologies for obtaining high-resolution portraits of tumor cellular ecosystems directly from primary tissue specimens. (See e.g., Jackson, H. W., et al. (2020). The single-cell pathology landscape of breast cancer. Nature 578, 615-620; Keren et al., 2019; Schürch, C. M., et al. (2020). Coordinated Cellular Neighborhoods Orchestrate Antitumoral Immunity at the Colorectal Cancer Invasive Front. Cell 182, 1-19; and Smith, E. A., and Hodges, H. C. (2019). The Spatial and Genomic Hierarchy of Tumor Ecosystems Revealed by Single-Cell Technologies. Trends in Cancer 5, 411-425; the disclosures of which are hereby incorporated by reference in their entireties.) However, practical considerations have largely limited these assays to single tumor types, modestly-sized sample cohorts, or small sets of phenotypic markers.

Here, we present an embodiment, referred to as EcoTyper, a new machine learning framework for delineating cell states and multicellular communities from primary tissues at unprecedented scale. Our approach combines statistical learning techniques with recent advances in gene expression deconvolution to illuminate multicellular communities in vivo from bulk tissue transcriptomes. (See Newman et al., 2019; cited above.) To demonstrate the utility of this new framework, we constructed a global atlas of transcriptionally-defined cell states from 16 types of human carcinoma. We then defined cell-state co-occurrence patterns across nearly 6,000 tumors, identifying 10 new multicellular communities with widespread representation. We validated our findings at the single-cell level, verified them in independent bulk tissue samples, and investigated their associations with genomic features, overall survival, and ICI response. Finally, we interrogated the spatial organization and developmental trajectories of two multicellular communities with proinflammatory properties. This work reveals fundamental units of cellular organization in human carcinoma, with implications for novel diagnostics and individualized therapies.

### Methods:

### Laser Capture Microdissection, Bulk RNA Sequencing, and IHC

4 μm full tissue sections of formalin-fixed paraffin-embedded (FFPE) CRC tumors (patients 380, 393, and 406) were prepared as previously described and areas of approximately 500 stromal cells were dissected using Arcturus XT LCM System. Sequencing libraries were prepared as described in “Smart-3SEQ starting from FFPE tissue on Arcturus LCM” protocol for HS caps as previously described and amplified for 22 PCR cycles. (See Foley, J. W., et al. (2019). Gene-expression profiling of single cells from archival tissue with laser-capture microdissection and Smart-3SEQ. Genome Research; the disclosure of which is hereby incorporated by reference in its entirety.) Library size distribution and concentration was assessed using Agilent 2200 TapeStation and qPCR with a dual-labeled probe. Libraries were sequenced using an Illumina NextSeq 500 instrument with High Output Kit v2.5, reading 76 bases for read 1 and 8 bases for read 2. Base calls from the NextSeq were demultiplexed and converted to FASTQ format with bcl2fastq (Illumina). The five-base unique molecular identifier (UMI) sequence and G-overhang were extracted from FASTQ data and A-tails were removed with umi_homopolymer.py (github.com/jwfoley/3SEQtools). Reads were aligned and further processed to remove duplicates using STAR (github.com/alexdobin/STAR). Bulk gene expression profiles were normalized to transcripts per million (TPM).

To confirm foamy macrophages by staining, 4 μm tissue sections of CRC patients 380 and 393 were deparaffinized, rehydrated, stained with H&E, and imaged at 20× magnification. Subsequently, slide coverslips were removed, and antigen retrieval was performed in EDTA pH 9 buffer for 5 min in 95° C. in a pressure cooker. Slides were next stained with CD68 XP monoclonal antibody (1/200, rabbit, Cell Signaling, D4B9C) and imaged at 20× magnification.

### Overview of EcoTyper Analytical Framework.

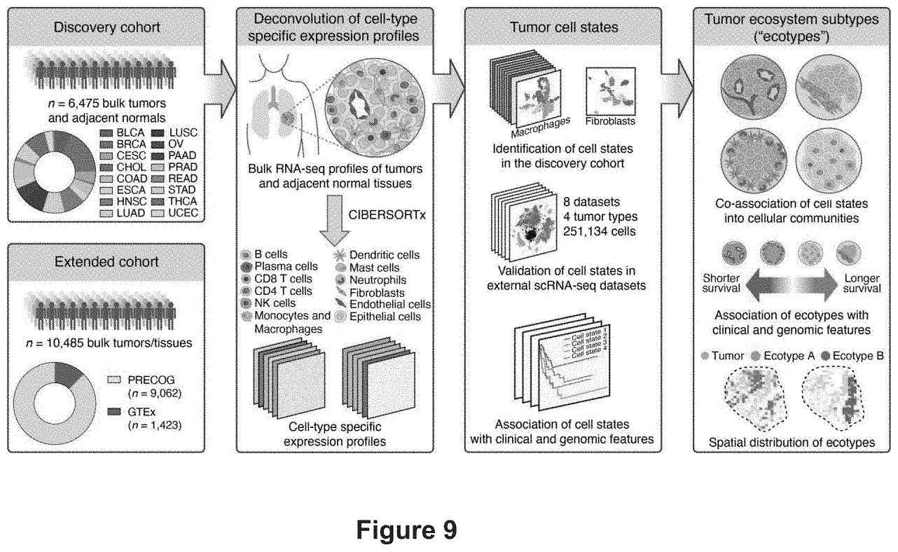

EcoTyper performs the following major functions, each graphically depicted in FIG. 9 with algorithmic details provided in the sections below.

In silico purification: Imputation of cell type-specific gene expression profiles from bulk tissue transcriptomes, at single-sample resolution.

Cell state discovery: Identification of recurrent cell type-specific transcriptional states.

Multicellular community discovery: Identification of multicellular communities through unsupervised clustering of cell-state co-occurrence patterns.

Cell state and community recovery: Recovery of cell states and communities in external expression data.

### In Silico Purification

To impute cell type-specific gene expression profiles from bulk tissue transcriptomes, EcoTyper employs CIBERSORTx, a recently described machine learning platform for digital cytometry. (See Newman et al., 2019; cited above.) Unlike related deconvolution methods, CIBERSORTx minimizes technical variation across platforms and can simultaneously purify expression profiles from multiple cell types (>10) at single-sample resolution. As input, CIBERSORTx requires a collection of optimized expression profiles that discriminate each cell type of interest, commonly termed a ‘signature matrix’. Signature matrices can be derived from single-cell or bulk-sorted transcriptomes and should be designed to cover major lineages within a particular tissue type. Once a signature matrix has been generated and validated, CIBERSORTx is applied to a dataset of uniformly processed bulk tissue transcriptomes to enumerate the frequencies of each cell type in the signature matrix. These estimates are then used to impute high-resolution cell type-specific gene expression profiles via a specialized implementation of non-negative matrix factorization with partial observations. Importantly, only genes with sufficient signal are imputed for each cell type, thereby minimizing the influence of spurious expression estimates on downstream results (Newman et al., 2019; Steen et al., 2020). The following equations and goals summarize the key CIBERSORTx steps used by EcoTyper:

B×F_{•,j}=M′_{•,j},1≤j≤n (1)

diag(G_{i,•,•}×F)=M_{i,•},1≤i≤g (2)

Given B, an m×c signature matrix consisting of m marker genes by c distinct cell types, and M′, an m×n matrix of bulk tissue gene expression profiles consisting of the same m genes by n samples, the goal of Equation 1 is to impute F, a c×n matrix consisting of the fractional abundances of c cell types for each sample in M′. (Note that M_{i,•} and M_{•,j} denote row i and column j of matrix M, respectively). Once F is imputed, the goal of Equation 2, which summarizes the high-resolution expression purification step of CIBERSORTx, is to impute G, a g×n×c matrix consisting of g genes, n samples, and c cell types, given F and M.

### Signature Matrix Design and Cell Fraction Estimation

To deconvolve 12 major cell types in human carcinomas (FIG. 9), we employed a hierarchical strategy in which two signature matrices, each previously validated in solid tumors (Newman et al., 2015; Newman et al., 2019; both cited above), were serially applied. First, major cellular compartments in epithelial tumors were deconvolved using TR4, a signature matrix consisting of epithelial (EPCAM+), endothelial (CD31+), fibroblast (CD10+), and bulk immune cell (CD45+) populations that were sorted from freshly resected surgical tumor samples from patients with NSCLC. (See e.g., Gentles, A. J., et al. (2015). The prognostic landscape of genes and infiltrating immune cells across human cancers. Nature Medicine 21, 938; the disclosure of which is hereby incorporated by reference in its entirety.) Through a series of benchmarking experiments, both from prior literature and the current work, we confirmed the high accuracy and generalizability of this matrix across multiple epithelial tumor types. To resolve leukocyte phenotypes, we employed LM22, a widely validated signature matrix consisting of 22 functionally-defined human hematopoietic cell types. We aggregated LM22 subsets into B cells, plasma cells, CD4 T cells, CD8 T cells, monocytes/macrophages, dendritic cells, natural killer (NK) cells, mast cells, and neutrophils. Because eosinophils were largely undetectable, they were excluded from further analysis. For the cell fraction enumeration step, CIBERSORTx was applied independently to each tumor type in the TCGA discovery cohort (FIG. 9) as previously described, using B-mode batch correction for LM22, no batch correction for TR4, no quantile normalization, and otherwise default parameters. To unify the signature matrices, leukocyte fractions from LM22 were rescaled to sum to 1 within each sample, then multiplied by total immune content imputed by TR4, yielding matrix F (Equation 1 above).

### Signature Matrix Validation

To validate the hierarchical strategy presented above, we created artificial tumor profiles using single-cell transcriptomes obtained from five scRNA-seq tumor atlases spanning three epithelial tumor types: NSCLC, CRC, and HNSC. From each scRNA-seq dataset, we simulated mixtures of defined fractions for the 12 cell types analyzed in this work (FIG. 9). First, we calculated means μ*₁, . . . μ*₁₂ and standard deviations σ*₁, . . . , σ*₁₂ for each cell type k₁, . . . , k₁₂ using fractions imputed by CIBERSORTx when applied to the same tumor type in the discovery cohort. Next, we sampled cell fractions from a gaussian distribution in which N(μ=μ*ᵢ, σ=max(0.25, 3σ*ᵢ)), for each cell type i. We then set negative fractions to 0 and re-normalized them to sum to 1 across all 12 cell types. Using the resulting fractions, we sampled 1,000 cells per dataset with replacement, aggregated their transcriptomes in non-log linear space into a pseudo-bulk mixture, then scaled the resulting mixture to TPM. Overall, 100 pseudo-bulk mixtures were created per dataset. CIBERSORTx deconvolution was applied to these mixtures as described above, but without batch correction.

### Expression Purification

To impute cell type-specific gene expression profiles, we provided two inputs to the high-resolution module of CIBERSORTx: the imputed fractions of all 12 cell types in the discovery cohort and a bulk expression matrix containing all tumor and adjacent normal samples (see External datasets—Discovery cohort). For this step, we restricted our analysis to protein coding genes, as annotated in GENCODE v24. High-resolution expression purification was run with default parameters, yielding matrix G (Equation 2 above).

To evaluate the cell type-specificity of purified expression profiles within G, we reanalyzed seven published scRNA-seq atlases of human carcinomas. First, we restricted scRNA-seq data to protein-coding genes (GENCODE v24). Next, we scaled each log₂-adjusted gene j to unit variance within each dataset. We then calculated, for each gene j, the log₂ fold change (FC) between each cell type (see External datasets—Single-cell RNA-seq tumor atlases) and the remaining cell types combined. Next, we averaged FCs for each cell type across the seven datasets and defined cell type-specific genes that satisfy the following three requirements: (i) FC of gene j is >0.1 in cell type i; (ii) FC of gene j is maximized in cell type i; (iii) 2nd highest FC of gene j is at least 0.1 lower than its maximum FC. We calculated pairwise Jaccard indices between detectably expressed genes imputed by CIBERSORTx and cell type-specific genes identified from scRNA-seq data. This process was repeated for each cell type, yielding a 12×12 Jaccard similarity matrix.

### Cell State Discovery.

EcoTyper leverages variants of nonnegative matrix factorization (NMF) to identify, recover, and validate transcriptionally-defined cell states from expression profiles purified by CIBERSORTx. Given c cell types, let Vᵢ←G_{•,•,i} be a g×n cell type-specific expression matrix for cell type i consisting of g rows (the number of genes) and n columns (the number of samples). The primary objective of NMF is to factorize Vᵢ into two non-negative matrices: a g×k matrix, W, and a k×n matrix, H, where k is a user-specified rank (i.e., number of clusters):

Vᵢ=W×H (3)

In EcoTyper, we employed NMF via Kullback-Leibler (KL) divergence minimization (Brunet et al., 2004; cited above), which starts with random initializations of the W and H matrices. This approach iteratively updates the following two equations until KL divergence is minimized:

$${W}_{gk} ← {W}_{gk} \frac{{Σ}_{n} [ {H}_{kn} {V}_{gn} / {( W \times H )}_{gn} ]}{{Σ}_{n} {H}_{kn}} \qquad ( 4 )$$

$${H}_{kn} ← {H}_{kn} \frac{{Σ}_{g} [ {W}_{gk} {V}_{gn} / {( W \times H )}_{gn} ]}{{Σ}_{g} {W}_{gk}} \qquad ( 5 )$$

Here, each cluster corresponds to a cell state. The basis matrix, W, encodes a representative expression level for each gene in each cell state. The mixture coefficients matrix H encodes the representation (relative abundance) of each cell state in each sample. Compared to alternative clustering approaches, NMF has three main advantages for cell-state discovery from digitally-purified transcriptomes. First, NMF naturally decomposes each expression profile into a set of constituent states. This sample-level decomposition is appropriate since expression profiles purified by CIBERSORTx are akin to bulk-sorted populations (e.g., CD4 T cells), which may contain multiple cell states in a given sample (e.g., naïve, memory, dysfunction CD4 T cells). Second, NMF identifies a set of states that best explain all purified expression profiles (for a given cell type) while simultaneously quantifying the relative abundance of each cell state. Third, NMF has analytical properties that enable assignment and validation of cell states in new data without re-training the model or deriving another classifier (see Cell state and community recovery).

EcoTyper applies NMF independently to each digitally-purified expression matrix Vᵢ produced by CIBERSORTx. For cell types with >1,000 detectably expressed genes, the top 1,000 genes with highest relative dispersion were selected as input. To do this for a given expression matrix Vᵢ, genes in log₂ space were averaged across samples and binned into 20 groups by 5 percentile increments. The relative dispersion of each gene was then calculated as the difference between its dispersion and the median dispersion of genes within the same expression bin, divided by the median absolute deviation of the dispersion of genes within the same expression bin.

Each gene was individually transformed to log₂ expression and standardized to unit variance within each tumor type. To satisfy the non-negativity requirement of NMF, cell type-specific expression matrices were individually processed using posneg transformation. This function converts an input expression matrix Vᵢ into two matrices, one containing only positive values and the other containing only negative values with the sign inverted. These two matrices are subsequently concatenated to produce V*ᵢ. The Brunet NMF algorithm implemented in the NMF R package version 0.20.0, with the n run parameter set to 1, was applied to Vᵢ* and run 50 times with different starting seeds. Among the 50 NMF models generated for a given rank and cell type, the model with the best fit, as determined by root mean squared error between V*ᵢ and the product of W and H, was selected. Each NMF mixture coefficients matrix, H, was rescaled such that the values in each column sum to 1 (i.e., each sample is represented as a mixture of cell state proportions that sum to 1 within each cell type). We interchangeably refer to the values in matrix H as cell state abundances or fractions. For analyses in which samples are assigned to specific cell states, each sample was assigned to the state with highest relative abundance among all states of a given cell type.

### Rank Selection

Cluster number selection is an important consideration in NMF applications. Previous approaches that rely on minimizing error measures (e.g., RMSE, KL divergence) or optimizing information-theoretic metrics either failed to converge or were dependent on the number of genes imputed (data not shown). Brunet and colleagues proposed a rank selection strategy based on evaluating the cophenetic coefficient, which quantifies the classification stability for a given rank (i.e., the number of clusters) and ranges from 0 to 1, with 1 being maximally stable. The rank at which the cophenetic coefficient starts decreasing is selected. This approach is challenging to apply in situations where the cophenetic coefficient exhibits a multi-modal shape across ranks, as we found for some cell types. Therefore, we developed a heuristic more suitable for such settings. In each case, the rank was chosen based on the cophenetic coefficient evaluated in the range 2-20 clusters, across 50 random restarts of the algorithm. Specifically, we determined the first occurrence in the interval 2-20 for which the cophenetic coefficient dropped below 0.95 (by default), having been above this level for at least two consecutive ranks, and selected the rank immediately adjacent to this crossing point which was closest to 0.95 (by default). We applied this approach for all cell types except two. First, for epithelial cells there was a steep drop in the cophenetic coefficient at rank 8, after which it stabilized just below 0.95. In this case, we chose rank 8. Second, for neutrophils, the cophenetic coefficient never decreased below 0.95; here we selected rank 5, the rank at which the cophenetic coefficient stabilized.

### Quality Control

We applied three quality control filters to eliminate non-robust states. First, we determined the number of ‘marker’ genes n in each state j with (i) nonzero expression in at least 50% of samples and (ii) a maximal log₂ fold change in state j relative to other states. States with n≤10 marker genes were omitted. Second, owing to the posneg transformation step noted above, NMF can identify states driven by features with more negative than positive values (after unit variance normalization). We hypothesized that such states are generally spurious. To test this, we derived a posneg ratio to flag these states, defined as the ratio between the sum of NMF weights from the W matrix corresponding to the positive features and the sum of weights corresponding to the negative features. Consistent with our hypothesis, states with a posneg ratio<1 were significantly less likely to be recoverable in scRNA-seq data (3.7% at P<0.05) as compared to those with a posneg ratio≥1 (85% at P<0.05) (see Cell state and community recovery below for the recovery procedure). We excluded all states with a posneg ratio<1, with the exception of one epithelial state (state S6) with a borderline posneg ratio (0.88) and >50 marker genes.

Finally, we identified poor-quality cell states using a dropout score, which flags states whose marker genes exhibit anomalously low variance and high expression across the discovery cohort. To calculate the dropout score for each marker gene (i.e., genes with maximal log₂ fold change in each state relative to other states within a given cell type), we determined the maximum fraction of samples for which the gene had the same value. We also calculated the average log₂ expression of the gene across samples. We averaged each quantity, scaled to unit variance across states, converted them to z scores, and removed states with a mean Z>1.96 (P<0.05).

In analyses involving discrete assignments of samples to cell states, samples that were assigned to discarded states were considered unassigned.

### Multicellular Community Discovery

To identify multicellular communities, we devised an approach in which pairwise co-associations between cell states were maximized while mutual avoidance within a cluster was minimized. First, we leveraged the Jaccard index to quantify the degree of overlap between each pair of cell states across tumor samples in the discovery cohort. Toward this end, we discretized each cell state q into a binary vector a of length l, where l=the number of tumor samples in the discovery cohort. Collectively, these vectors comprised binary matrix A, with 69 rows (states)×1 columns (samples). Given sample s, if state q was the most abundant state among all states in cell type i, we set A_{q,s} to 1; otherwise A_{q,s}←0. We then computed all pairwise Jaccard indices on the rows (states) in matrix A, yielding matrix J with 69 rows×69 columns. Using the hypergeometric test, we evaluated the null hypothesis that any given pair of cell states q and k has no overlap. In cases where the hypergeometric p-value was >0.01, the Jaccard index for J_{q,k} was set to 0 (i.e., no overlap). To identify communities while accommodating outliers, the updated Jaccard matrix J′ was hierarchically clustered using average linkage with Euclidean distance (hclust in the R stats package). The optimal number of clusters was then determined via silhouette width maximization. Clusters with ≤2 cell states were eliminated from further analysis, leaving 10 clusters, which we termed carcinoma ecotypes (CEs).

To evaluate the robustness of CE definitions, we applied two alternative methods to J′: Louvain community detection and k-medoids. To evaluate the Louvain algorithm (NetworkToolbox v1.4.0 package in R), we determined the set of parameters, gamma, that produced each number of clusters between 2 and 30 and selected the value of gamma that produced the number of clusters with the highest mean silhouette width. To evaluate k-medoids (pam function in the R package, cluster v2.0.7), we varied the number of centroids between 2 and 30 and selected the number that maximized the mean silhouette width. To promote a fair comparison, we filtered out communities with less than three states (as above), then rendered the comparisons as river plots.

To estimate CE abundance, cell state abundances from each CE were averaged. The resulting values were normalized to sum to 1 across all CEs in each sample. To assign samples to CEs, we applied a two-sided t test with unequal variance to evaluate the difference in estimated abundance between the cell states from each CE relative to the remaining CEs. The resulting p-values were corrected for multiple hypothesis testing using the Benjamini-Hochberg method. Each sample was assigned to the CE with the highest CE abundance if: (i) its corresponding q-value was less than 0.25 and (ii) the sample was assigned to at least one cell state contributing to CEs. Otherwise, the sample remained unassigned. For melanoma datasets, epithelial cell states were ignored when determining CE assignments.

### Cell State and Community Recovery

We leveraged the internal structure of the NMF model to devise a reference-based strategy for recovering cell states in new samples.

### Quantitation

In classical NMF, matrices W and H are iteratively updated according to Equations 4 and 5 until convergence. In a new dataset, V′, one can reuse the previously fit cell type-specific basis matrix W in order to determine the mixture coefficients matrix H′ in new samples:

V′=W×H′ (6)

This update procedure consists of iteratively updating H′ until convergence of Equation 6. This approach has three distinct advantages over alternative methods for supervised classification. First, the mathematical structure of the original model is maintained when classifying new samples. This eliminates the need to train another classifier and avoids the introduction of new assumptions or biases that lead to information loss. Second, this approach mirrors the output of the original NMF model, facilitating consistent interpretation. Third, unlike methods that perform supervised classification independently for each sample, the matrix H′ is jointly updated across all samples, increasing the robustness of cell state recovery.

We implemented this framework within EcoTyper and applied it to external expression datasets analyzed in this work (FIG. 9). In each case, EcoTyper solved Equation 6 using the cell type-specific NMF models defined in the discovery cohort. Prior to analysis, each gene was log₂-transformed and scaled to unit variance within each tumor type (TCGA/PRECOG), healthy tissue type (GTEx), spatial transcriptomics array, or individual dataset (scRNA-seq data, immunotherapy datasets, and early tumor development datasets), as appropriate. Once cell states were quantitated, multicellular community abundance was determined, as described in Multicellular community discovery. To assess the accuracy of cell state recovery, we solved Equation 6 on all bulk tumor transcriptomes in the discovery cohort. Cell-state fractions were concordant with those obtained on expression profiles derived from digitally-purified expression profiles (CIBERSORTx), demonstrating successful sample decomposition.

### Statistical Significance

To determine the statistical significance of reference-guided cell state recovery from scRNA-seq data, we devised a permutation-based approach. First, we assigned single-cell transcriptomes to cell states by solving Equation 6. Each cell of a given cell type was assigned to the state with maximum weight. Next, for each state s and its corresponding list of predefined marker genes gₛ (same as those defined in Quality control step 1, but without the percent expression filter), we calculated—for each gene j in gₛ—the average fold change between the cells assigned to state s and the cells assigned to other states, obtained after log₂ transforming the normalized counts and scaling to zero mean and unit variance across cells. The average fold change, AFCₛ, of marker genes gₛ in state s was compared with the corresponding 100 values, AFC_{s,i}^{shuffled}, 1≤i≤100 obtained by independently shuffling the expression values of each gene in gₛ across all cells and solving Equation 6 one hundred times. We then calculated a z-score to quantify the probability that AFCₛ is significantly higher than AFC_{s,i}^{shuffled}, using the formula:

$${z}_{s} = \frac{{AFC}_{s} - mean ( {AFC}_{s , i}^{shuffled} )}{sd ( {AFC}_{s , i}^{shuffled} )}$$

For scRNA-seq datasets where more than 2,500 cells from a particular cell type were available, the procedure was applied on a set of 2,500 randomly selected cells. This was done to mitigate imbalances in the number of cells per cell type and for the sake of computational efficiency. However, even without this step, results were comparable (data not shown). The resulting z-scores were combined across scRNA-seq datasets using Stouffer's method and converted to one-sided p-values.

### Discovery Cohort

First, to focus on tumor samples of epithelial origin, we excluded brain cancers (GBM, LGG), blood cancers (DLBC, LAML, LCML), sarcomas (SARC, UCS), and melanomas (SKCM, UVM). Second, we tested whether housekeeping genes were uniformly expressed across tumor types. By performing hierarchical clustering (hclust in the R stats package with complete linkage and Euclidean distance) on the log₂ expression matrix of 11 previously defined housekeeping genes, we identified two robust clusters using the silhouette method. The minority cluster, which consisted of five tumor types (ACC, KICH, KIRC, KIRP and PCPG), was excluded. Next, we tested whether CIBERSORTx coupled with TR4 (see ‘Signature matrix design and cell fraction estimation’) could reliably impute epithelial composition across tumor types via comparison to ESTIMATE. Although Pearson correlation coefficients between the two methods were generally high (r>0.8 for 90% of tumor types), mean squared errors (MSEs) were variable. Using K-means and silhouette maximization to jointly cluster Pearson correlation coefficients and MSEs, we identified a single outgroup with high MSE which we omitted from further analysis. Finally, to mitigate the influence of technical variation on deconvolution results, we removed samples if they were (i) flagged as poor quality by prior studies or (ii) generated on an Illumina platform other than HiSeq2000 v2, which encompassed ˜85% of the remaining evaluable tumors. The final discovery cohort, which was uniformly processed and standardized, consisted of 16 carcinomas spanning 5,946 tumor and 529 adjacent samples.

### Single-Cell RNA-Seq Tumor Atlases

We compiled and curated scRNA-seq tumor atlases from seven datasets covering breast carcinoma, head and neck squamous cell carcinoma (HNSC), colorectal cancer (CRC), non-small cell lung cancer (NSCLC), and melanoma. All datasets were pre-processed and scaled to TPM or counts per million (CPM), as appropriate. Author-supplied cell type assignments were used with the following exceptions:
In the breast cancer dataset of Azizi and colleagues, cells labeled as regulatory T cells were grouped with total CD4 T cells.
In the colorectal dataset of Park and colleagues, the authors' clusters were mapped to cell types using the schema in Table S1.
In the HNSC dataset of Puram and colleagues and the melanoma dataset of Tirosh and colleagues, T cells were not divided into CD8 and CD4 T cells by the authors. Thus, we annotated them using the FindClusters function in Seurat v2.3.4, applied on the first 20 principal components of each dataset, with the resolution parameter set to 0.1, and other parameters set to default. In both datasets, CD8 and CD4 T cell clusters distinguished by high expression of CD4/IL7R and CD8A/CD8B, respectively, were resolved.
In the NSCLC dataset of Lambrechts and colleagues, cell clusters identified by the authors were mapped to phenotypic labels as follows: For clusters defined in Lambrechts et. al., clusters 1, 2, 5 and 7 were assigned to B cells, clusters 3 and 6 to plasma cells, and cluster 4 to mast cells. For clusters defined in FIG. 4f of Lambrechts et. al., clusters 1, 2, 3, 4, 6, 8, 10, 11 were assigned to monocytes/macrophages, clusters 5, 9, 12 to dendritic cells, and cluster 7 to neutrophils. For clusters defined in FIG. 5b of Lambrechts et. al., clusters 2, 4, 5, 8 were assigned to CD8 T cells, clusters 1, 3, 9 to CD4 T cells, and cluster 6 to NK cells.
In the NSCLC dataset of Laughney and colleagues, cells annotated as “Breg” were assigned to B cells; “IG” to plasma cells; “NK” and “NKT” to NK cells; and “Th”, “Tm” and “Treg” to T cells. T cells were subdivided into CD4 and CD8 T cells using the FindClusters function in Seurat v2.3.4, applied on the first 20 principal components, with the resolution parameter set to 0.1, and other parameters set to default.
In the NSCLC dataset of Zilionis and colleagues, CD4 T cell subsets, dendritic cell subsets, and monocyte/macrophage subsets were merged into their corresponding parental lineages. Only cells collected from tumors were analyzed

### Clinically-Annotated Bulk Tumor Transcriptomes

We analyzed 9,062 pre-normalized carcinoma transcriptomes from the Prediction of Cancer Outcomes using Genomic Profiles (PRECOG) database, along with additional datasets, all of which were processed according to the PRECOG workflow. All datasets (n=35) were filtered to only include malignancies with matching tumor types in the discovery cohort and with at least 100 samples and available overall survival data.

### Healthy Tissue Transcriptomics

Raw feature counts for GTEx samples (GSE86354) were downloaded and filtered to retain seven distinct tissue types, each of which was selected as a normal tissue counterpart for a tumor type in the discovery cohort. To address differences in normalization between TCGA and GTEx, we integrated and co-normalized the discovery cohort and GTEx using the following procedure. First, we merged count matrices from TCGA (GSE62944) and GTEx, applied upper quartile normalization using the EDASeq package in R, calculated CPM, then log₂-transformed the data. We then determined the unit variance scaling parameters specific for each gene in each TCGA tumor type necessary to bring the corresponding GTEx tissue type into the same space. Specifically, for a given gene g, we calculated its mean μ_{gt} and variance σ_{gt} across tumor samples from tumor type t. Then, the log₂ expression level e_{gs} of gene g in the GTEx sample s, from the tissue matching the tissue-of-origin for tumor type t, was normalized using the formula:

$${e}_{gs}^{new} ← \frac{{e}_{gs} - {μ}_{gt}}{{σ}_{gt}}$$

The resulting set of 1,423 normalized GTEx samples was used for further analyses.

### Immunotherapy

For analyses related to ICI response, we downloaded clinically-annotated bulk tumor transcriptomes from patients with metastatic urothelial carcinoma (bladder cancer) and metastatic melanoma. The former was generated by the IMvigor210 phase II atezolizumab trial and obtained via the R library Imvigor210CoreBiologies version 1.0.0. Raw read counts were converted to TPM. For the latter, normalized count data and clinical annotations were downloaded and converted to TPM.

### Single-Cell Validation of Cell States Enriched in Known Phenotypes

To determine whether states enriched in adjacent normal tissue can be recapitulated at the single-cell level, we used an NSCLC scRNA-seq atlas containing cells from tumors and adjacent normal tissues. We started by restricting our analysis to NSCLC adenocarcinoma and squamous cell carcinoma histological subtypes (LUAD/LUSC in the discovery cohort). We then tested the null hypothesis that the number of adjacent normal samples in the discovery cohort and the number of single cells from adjacent normal specimens in the scRNA-seq dataset, assigned to each cell state, are lower than or equal to the respective numbers obtained by random chance. Specifically, we counted the number Nₛ of adjacent-normal samples/cells assigned to state s by Ecotyper. Then, for 1,000 iterations, we calculated the number N_{s,i}^{shuffled} of adjacent normal samples/cells assigned to state s after randomly permuting the cell state assignment labels at iteration i, thus generating a null distribution. Based on this distribution, we calculated a z-score:

$${z}_{s} = \frac{{N}_{s} - mean ( {N}_{s , i}^{shuffled} )}{sd ( {N}_{s , i}^{shuffled} )}$$

Z-scores were converted to two-sided p-values and states with P<0.05 were considered significantly enriched in adjacent normal samples. We applied this approach to the same datasets, but focused on adenocarcinoma versus squamous cell carcinoma samples/cells.

Identification of State-Specific Marker Genes in scRNA-Seq Data

The number of genes imputed by CIBERSORTx is adaptively determined for each cell type. To both extend the number of marker genes assigned to each state and assess robustness, we used a reference-guided approach to map single-cell transcriptomes to EcoTyper states, as described above (Cell state and community recovery). This was done for every scRNA-seq atlas utilized in this work. For each gene g and state s, we then considered the following criteria for prioritizing marker genes from scRNA-seq data:
The number of scRNA-seq datasets n₁ in which g is expressed (i.e., mean TPM/CPM>0)
The number of scRNA-seq datasets n₂ for which g is assigned to state s
The quantity n₃←n₂/n₁
The number of distinct tumor types n₄ for which g is assigned to state s for at least one scRNA-seq dataset from each tumor type
The number of scRNA-seq datasets n₅ for which g is significantly differentially expressed using Seurat (Q<0.05)
The quantity n₆←−log₁₀(MetaQ_{g,s}), where MetaQ_{q,s} is defined as an aggregate p-value for differential expression of gene g in state s across all evaluable scRNA-seq datasets, adjusted for FDR as detailed below
The quantity n₇←AvgFC_{g,s}, where AvgFC_{g,s} is defined as the mean log₂ fold change of gene g in state s within each evaluable scRNA-seq dataset, aggregated by mean across all evaluable datasets

For each state s, the above quantities {n₁, n₂, . . . , n₇} were converted to rank space and averaged across measures, yielding a composite score for each gene g, denoted S_{g,s}. We combined manually curated genes with the top marker genes selected by decreasing S_{g,s}.

Prior to calculating the seven quantities above, for each scRNA-seq dataset d and cell type i, we excluded cell states with <5 assigned cells along with the cells mapping to them. Genes were assigned to cell states based on the state with the maximum log₂ fold change relative to other states, across scRNA-seq datasets. Ties were broken by the state for which the gene had the highest average log₂ fold change. Genes were excluded if the assigned state differed from the state identified by EcoTyper. To calculate n₅, we used FindMarkers function in Seurat, with min.pct=0.1 and log fc·threshold=0.05. To calculate n₆, we converted the nominal (unadjusted) p-values calculated by Seurat into two-sided z-scores, with the direction determined by the orientation of the fold change of gene g in state s. We then aggregated z-scores across datasets by Stouffer's method, converted the resulting meta-z scores to two-sided p-values, and adjusted the resulting p-values for multiple hypothesis testing via the Benjamini-Hochberg procedure, yielding MetaQ_{q,s}.

Leave-One-Out Cross-Validation of scRNA-Seq Markers

To assess the robustness of the top markers selected as described above, we employed the following leave-one-out cross-validation (LOOCV) procedure. Briefly, we applied the above-mentioned marker selection strategy to all scRNA-seq datasets except one, and for each cell type k and state s, we assessed the top 10 marker genes, as defined by decreasing score S_{g,s}, in the held-out dataset. To do this, we first scaled each gene in the held-out dataset to log₂ expression and unit variance across all cells mapping to cell type k. For each state i in k, we calculated the mean expression of each gene and averaged these values across the 10 marker genes, yielding AvgS_{k,i}. We then determined the state s′ in which

$${s}^{′} = {max}_{i} ( {AvgS}_{k , i} ) .$$

We tallied all states for which s′=s, then repeated this process for all held-out datasets, yielding a LOOCV rate for each state s. Across all states, the mean LOOCV was 90%.

### Lineage-Specific Differences in Cell State Composition

We derived a tumor specificity index for each cell type. First, for each tumor type in the discovery cohort, we calculated the fraction of tumor samples assigned to each cell state q of a given cell type k. Discrete assignments were made as described in ‘Multicellular community detection’ above. This process produced a matrix of fractions F, consisting of 69 states×16 tumor types. Next, for each cell type k, we extracted the subset of F corresponding to cell states in k (denoted Fₖ) and calculated the Pearson correlation matrix Pₖ across all columns in Fₖ. We then calculated the mean of the upper triangle of Pₖ, denoted μₖ. The tumor specificity index of each cell type k was calculated as 1−μₖ.

### In Silico Annotation of Monocyte and Macrophage States

We assembled previously normalized whole transcriptome data of human monocyte and macrophage subsets, including classical M0 macrophages and polarized M1/M2 macrophages. For each cell subset, we rank-ordered each gene in the transcriptome by calculating the average log₂ fold change of each cell type relative to the others. To incorporate foamy cell macrophages into this analysis, we used a previously published differential expression analysis of foamy vs. non-foamy cell macrophages isolated from ApoE null mice by differential plastic adherence. Mouse gene symbols (GRCm38.p6) were converted to homologous human gene symbols (GRCh38.p13) using BioMart v2.38. We evaluated the top 50 marker genes (defined in Identification of state-specific marker genes in scRNA-seq data) of each monocyte/macrophage state in mean log 2 fold change-ordered transcriptomes using pre-ranked Gene Set Enrichment Analysis (GSEA) (fgsea, from fgsea package), with 10,000 permutations.

### Survival Analyses and Response to Therapy

We applied univariate Cox proportional hazards regression to link the relative abundance of each cell state (or CE) to overall survival. This was done separately for each tumor type and dataset. The resulting z-scores were integrated across datasets of the same tumor type using Liptak's method with weights set to the inverse of the Cox model coefficient standard errors. Meta-z scores were further combined across tumor types using Stouffer's method. To assess the association of each cell state and CE with overall survival after multivariate adjustment for age, sex, and pathologic stage, Cox regression was applied to (i) the relative abundance of each cell state (or CE), (ii) age as a continuous variable, (iii) sex as a binary variable, and (iv) stage as a categorical variable. Multivariate models were fit for each tumor type separately and global meta-z-scores were calculated using Stouffer's method. All survival z-scores were converted to two-sided −log₁₀ p-values for clarity.

To obtain the Kaplan Meier plots, we started by calculating the difference vector (denoted d) between the imputed abundances of monocyte/macrophage states 6 and 3. To identify a threshold in d that maximally stratifies overall survival, we divided d into 20 possible cut-points at even 5 percentile intervals within tumor types in the discovery cohort. We then determined the hazard ratio and log-rank p-value for each potential cut-point. Next, we converted the hazard ratios and −log₁₀ p-values to rank space and determined the value b with highest mean rank. For each tumor type, we optimized b in the discovery cohort and used it to stratify survival curves in the discovery (TCGA) and validation (PRECOG) cohorts.

We evaluated the following candidate correlates of ICI response:
Cell state and CE abundance vectors predicted by EcoTyper (n=69 and 10, respectively).
Log₂ expression levels of CD8A, PDCD1, CTLA4, and IFNG, each scaled to unit variance across all pretreatment samples in each dataset.
CIBERSORTx proportions of epithelial cells (bladder cancer only), melanoma cells (melanoma datasets only), fibroblasts, endothelial cells, the 9 immune cell types in FIG. 1, and LM22 subsets not covered in FIG. 1. Immune subsets were evaluated scaled to total immune content and scaled relative to all non-redundant cell types.
   IMvigor210 dataset: CIBERSORTx was run with LM22 and TR4 signature matrices, as described above (see Signature matrix design and cell fraction estimation).
   Melanoma datasets: CIBERSORTx was run with LM22 (B-mode batch correction) and a previously described scRNA-seq-based signature matrix covering melanoma cells, fibroblasts, endothelial cells, and immune subsets from melanoma tumor biopsies (B-mode batch correction). Immune cell fractions in the latter were replaced with LM22 in order to scale LM22 fractions into absolute space.
Tumor mutational burden (TMB) were used as supplied by the authors: the number of nonsynonymous mutations per sample, the number of point mutations per sample, the number of neoantigens per sample, and neoantigen burden per MB. (See Riaz, N., et al. (2017). Tumor and Microenvironment Evolution during Immunotherapy with Nivolumab. Cell 171, 934-949.e916; Van Allen, E. M., et al. (2015). Genomic correlates of response to CTLA-4 blockade in metastatic melanoma. Science 350, 207-211; Nathanson, T., et al. (2017). Somatic Mutations and Neoepitope Homology in Melanomas Treated with CTLA-4 Blockade. Cancer Immunology Research 5, 84-91; Liu, D., et al. (2019). Integrative molecular and clinical modeling of clinical outcomes to PD1 blockade in patients with metastatic melanoma. Nature Medicine 25, 1916-1927; and Mariathasan, S., et al. (2018). TGFβ attenuates tumour response to PD-L1 blockade by contributing to exclusion of T cells. Nature 554, 544-548; the disclosures of which are hereby incorporated by reference in their entireties.)
Previous signatures of ICI response and/or T cell cytotoxicity:
   Immune resistance program score, calculated using code supplied by the authors (ImmRes_OE.R script, run using the TPM matrix of each dataset as input and parameter sig set to res.sig object from the resistance.program.RData environment). (See Jerby-Arnon, L., et al. (2018). A Cancer Cell Program Promotes T Cell Exclusion and Resistance to Checkpoint Blockade. Cell 175, 984-997.e924; the disclosure of which is hereby incorporated by reference in its entirety.)
   18-gene T cell-inflamed score. The log₂ expression values of each gene were scaled to unit variance across samples, and the resulting scaled values were averaged within each sample. (See Cristescu, R., et al. (2018). Pan-tumor genomic biomarkers for PD-1 checkpoint blockade-based immunotherapy. Science 362, eaar3593; the disclosure of which is hereby incorporated by reference in its entirety.)
   Cytolytic score, calculated as the geometric mean of GZMA and PRF1. (See Rooney, M. et al. (2015). Molecular and Genetic Properties of Tumors Associated with Local Immune Cytolytic Activity. Cell 160, 48-61; the disclosure of which is hereby incorporated by reference in its entirety.)

All ICI expression datasets were TPM normalized prior to analysis. Only RNA-seq profiles of pretreatment tumors were analyzed. Each of the above measures was estimated independently in each dataset to avoid possible batch effects. We applied univariate Cox proportional hazards regression to each measure and extracted the z-score capturing its association with overall survival. We also assessed each measure's binary association with response to therapy using a two-sided Wilcoxon test, from which we calculated a z-score from the Wilcoxon p-value. Z-scores were integrated across datasets by therapy type (aPD1, aPD1, or aCTLA4) using Liptak's method, with the number of samples as weights. The ranks of the resulting z-scores were calculated for each combination of outcome association and therapy type and then averaged to yield a final rank for each measure.

### CE Network Visualization

For network schematics, weighted undirected networks, representing the cell states from each CE were constructed using the igraph package, version 1.2.2. The edge weights were proportional to the Jaccard index between cell states, and the layout of each network was generated using the forced directed layout algorithm by Fruchterman and Reingold, implemented in the layout_with_fr function. (See Fruchterman, T. M. J., and Reingold, E. M. (1991). Graph drawing by force-directed placement. Software: Practice and Experience 21, 1129-1164; the disclosure of which is hereby incorporated by reference in its entirety.)

### Ligand-Receptor Enrichment Analysis

To determine whether CEs enrich for potential heterotypic interactions, we compiled a list of ligand-receptor pairs and assessed their statistical enrichment in each CE. We started by determining CE-specific differential expression. For each cell state of a given CE i, we assessed whether each gene, scaled to unit variance within each tumor type, was overexpressed in samples assigned to CE i relative to samples assigned to the remaining CEs. This was done using digitally-purified expression profiles produced by CIBERSORTx. Statistical significance was determined using a two-sided t-test with unequal variance corrected for multiple hypothesis testing (Benjamini-Hochberg). Genes with a q-value<0.05 and log₂ fold-change>0.1 were considered significant. Next, for each unique pair of states q,s within CE i, we calculated the number of putative ligand-receptor pairs, Ir_{ref}, for which the ligand was over-expressed in cell state q and the receptor over-expressed in cell state s. We compared the number of putative ligand-receptor pairs in states q, s against a null distribution of 100 samples, lrₛₐₘₚ^{1 . . . 100}, obtained by drawing gᵢ and g₂ genes from list l, where g₁ is the number of genes over-expressed in state q; g₂ is the number of genes over-expressed in state s; and l is the non-redundant set of genes among experimentally-determined ligand/receptor pairs that overlap genes imputed by CIBERSORTx in states q and s. We obtained a two-sided z-score for each pair of cell states q,s using the following formula:

$${z}_{q , s} = \frac{{lr}_{ref} - mean ( {lr}_{samp}^{1 … 100} )}{sd ( {lr}_{samp}^{1 … 100} )}$$

To obtain a CE-level measure of enrichment, we integrated individual z-scores for all pairwise state comparisons within CE i using Stouffer's method.

Statistical Significance of CE Recovery in scRNA-Seq Data

To determine whether CEs are detectable at the single-cell level, we analyzed six scRNA-seq tumor atlases that collectively cover 97 tumor and adjacent normal tissues and samples and all major cell types analyzed in this work. We calculated significance at the level of individual CEs (CE-specific probability of detection) and across all CEs simultaneously (Joint probability of CE detection). In both cases, we assigned single-cell transcriptomes to EcoTyper states blinded to CE identity (see Cell state and community recovery). We also calculated, for each tumor or adjacent normal sample i, the fraction of each cell state j within each parental cell type, yielding matrix F, with 58 rows (i.e., the number of cell states within CEs) and 97 columns (i.e., the number samples). To mitigate the impact of distortions in state representation due to tissue dissociation, noise, dropout, and under-sampling, we devised a co-occurrence index that integrates four alternative approaches for correlating cell-state abundance profiles via ensemble averaging. First, we calculated two versions of F with different denominators for calculating cell-state abundance: one version limits the denominator to the set of cells that could be assigned to EcoTyper states (F₁); the other does not (F₂). Next, we calculated four spearman correlation matrices: Two matrices were calculated directly from the rows of F₁ and F₂, yielding matrices C₁ and C₂; the others were calculated after replacement of zeros in F₁ and F₂ with NA, yielding C₃ and C₄. The latter provides robustness to under-sampling of cell states within individual tumor or normal tissue samples. We averaged the four matrices into matrix C with 58 rows×58 columns.

### Probability of CE Detection

We implemented a permutation-based approach to determine whether cell states within a given CE co-associate more strongly than expected by random chance. First, we set all diagonal entries in C to NA. Next, for each CE i and cell state j, we calculated the mean co-occurrence index between state j and the other states in CE i, denoted α₁, and the mean co-occurrence index between state j and all other states in the remaining CEs, denoted μ₂. We then calculated Δᵢ as μ₁-μ₂ and repeated this process for all states with CE i. The test statistic for CE i is mean(Δ), denoted AvgDif_{ref}. To derive a null distribution, we permuted each row of C 1,000 times, each time repeating the above procedure. For each row in the permuted matrix, we swapped NA (diagonal entry in the original matrix) with the new diagonal entry prior to calculating mean(Δ). This yielded the null distribution, AvgDif_{shuf,i}. We calculated the significance of CE i according the following formula:

$${z}_{i} = \frac{{AvgDif}_{ref} - mean ( {AvgDif}_{shuf , i} )}{sd ( {AvgDif}_{shuf , i} )}$$

Z-scores were converted to P values for ease of interpretation.

### Joint Probability of CE Detection

To obtain a global statistic for the joint probability of CE detection in scRNA-seq data, we applied the above approach (Probability of CE detection) with modifications. Specifically, for each CE i and cell state j, we calculated the mean co-occurrence index between state j and the other states in CE i, denoted μᵢ, and repeated this process after permuting the rows (as above) to calculate μ^{shuf}ᵢ. We then counted the number of CEs (out of 10) with μᵢ≥μ^{shuf}ᵢ for all i.

### Feature Analysis of Carcinoma Ecotypes

We compiled and curated pre-computed data covering genomic characteristics and mutational signatures in TCGA tumors. We also analyzed Hallmark gene sets from MSigDB and physiological variables. Continuous and discrete features were analyzed separately. For continuous features, we analyzed bulk tumors based on their CE assignment. To incorporate Hallmark gene sets, we averaged all component genes in log₂ space after scaling each gene to unit variance expression across samples. Enrichment/depletion of each feature across CEs was calculated by performing a two-sided Wilcoxon test to compare the sample-level values of each feature in a CE relative to other CEs. The resulting p-values were adjusted for multiple hypothesis testing across all evaluated features using the Benjamini-Hochberg method. Features with a q-value<0.05 were considered significantly enriched/depleted. The magnitude of enrichment/depletion was calculated as the difference in the average value of each feature across samples within a given CE relative to other CEs. For discrete features (i.e., sex; age binarized as ≥60 or <60 years), CE-specific associations were determined by applying a two-sided Wilcoxon test to compare the relative abundance of each CE between groups (e.g., male vs. female). P-values were adjusted for multiple hypothesis testing as described above. The magnitude of the enrichment/depletion was calculated as the average CE abundance within each group versus the other group (e.g., ≥60 vs. <60 years).

State-Specific Expression in CE9 and CE10 Across scRNA-Seq Datasets

For each scRNA-seq dataset, differential expression analysis was performed between CE9 and CE10-specific cell states, for each cell type with states in both ecotypes, using Seurat v3.1.3. Count data were log₂-adjusted using NormalizeData with default parameters. For each cell type, differentially expressed genes between CE9- and CE10-specific states were identified using FindMarkers with min.pct=0.1 and log fc·threshold=0.05. To integrate across datasets, for each gene in a cell type, nominal p-values were converted to z-scores and z-scores were combined across scRNA-seq datasets using Stouffer's method. Meta z-scores were then converted to p-values which were corrected by the Benjamini-Hochberg method. Genes with a q-value<0.25 and with positive expression in at least 10% of cells in CE9 or CE10 were considered differentially expressed. If <10 genes passed this filter, we selected marker genes from the table described in ‘Identification of state-specific marker genes in scRNA-seq data’. To admit genes from this table, we required a q-value<0.25, average log₂ fold change>0.1, and average non-zero expression in at least 10% of cells in CE9 or CE10, across scRNA-seq datasets.

Once significant differentially expressed genes were identified, we selected the top 75 genes by average log₂ fold change for each cell state, or the minimum between the number of marker genes in CE9 and CE10 states if less than 75 were available. Within each scRNA-seq dataset, the final list of genes was log₂ adjusted and unit variance-normalized across cells, then averaged by ecotype. Prior to plotting, we applied unit variance normalization across genes to mitigate dataset-specific variation in the magnitude of expression.

### Results:

### The EcoTyper Framework

We designed EcoTyper as a broadly applicable framework for high-throughput identification of cell states and multicellular communities from primary tissue specimens. It consists of three key steps: digital purification of cell type-specific gene expression profiles from bulk tissue transcriptomes, identification and quantitation of transcriptionally-defined cellular states, and co-assignment of cell states into multicellular communities (FIG. 1).

EcoTyper starts by applying CIBERSORTx, a recently described approach for ‘digital cytometry’, to determine the abundance and gene expression profiles of individual cell types within bulk tissue transcriptomes. By imputing the composition of major cell types within a collection of related tissue specimens, CIBERSORTx can mathematically purify gene expression profiles for multiple cell types of interest without single-cell sequencing or physical cell isolation. Second, EcoTyper employs statistical learning algorithms, including variants of non-negative matrix factorization, to identify cell type-specific transcriptional programs (“cell states”), quantify their relative representation in each sample, and recover them in external expression datasets. Third, EcoTyper determines co-occurrence patterns between cell states that define multicellular communities. Once defined, EcoTyper can query cell states and communities across datasets and platforms, allowing for large-scale assessment of the composition, signaling pathways, spatial topology, and clinical correlates of cellular ecosystems.

### Atlas of Transcriptionally-Defined Cell States in 16 Carcinomas

To demonstrate the utility of EcoTyper, we used it to gain insights into human carcinoma, the leading cause of cancer deaths worldwide and a class of malignancies for which extensive genomic and clinical data are publicly available. As carcinomas originate from epithelial cells, we started by selecting 12 cell types that together span the majority of immunological and structural cells found in human epithelial tumors: B cells, plasma cells, CD8 T cells, CD4 T cells, NK cells, monocytes/macrophages, dendritic cells, mast cells, neutrophils, fibroblasts, endothelial cells, and epithelial cells. We then assembled a collection of cell type-specific gene expression signatures to discriminate each cell type using CIBERSORTx. For this purpose, we took advantage of previously published gene expression signatures, each with extensive validation data supporting their analytical performance for deconvolving solid tumors, including carcinomas.

Next, we compiled a discovery cohort consisting of 16 types of human carcinoma spanning 5,946 tumor and 529 adjacent normal transcriptomes profiled by The Cancer Genome Atlas (TCGA) (FIG. 9). (See Tatlow, P. J., and Piccolo, S. R. (2016). A cloud-based workflow to quantify transcript-expression levels in public cancer compendia. Sci Rep 6, 39259; the disclosure of which is hereby incorporated by reference in its entirety.) These datasets were selected to maximize the consistency of specimen handling and processing, the accuracy of imputed cell fractions against orthogonal measures, the uniformity of expression levels across diverse housekeeping genes, and the availability of both genomic data and clinical follow-up for each biospecimen. Applied to these data, which were uniformly processed and standardized, EcoTyper produced a matrix of 150 million data points representing 77,700 digitally-purified expression profiles, one for each evaluated cell type and patient sample (i.e. 12 cell types×6,475 samples).

The size and scope of this expression matrix provided an unprecedented opportunity to identify and validate tumor-associated cell states that are shared across cancers. First, we confirmed that all profiles showed strong evidence of cell type-specificity by comparison to reference profiles derived from scRNA-seq data. Next, we applied EcoTyper to model each digitally-purified sample as a linear combination of discrete transcriptional programs. In this way, purified samples were treated as bulk-sorted populations, allowing multiple transcriptional states to coexist per sample.

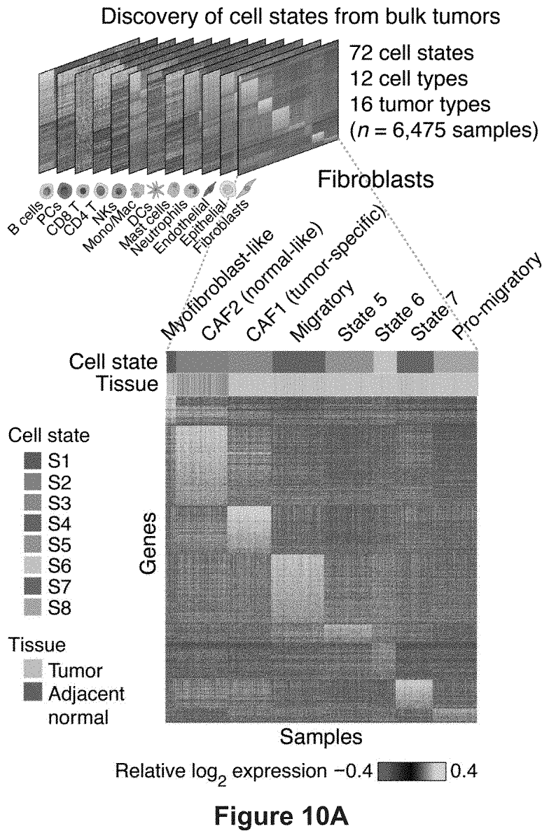

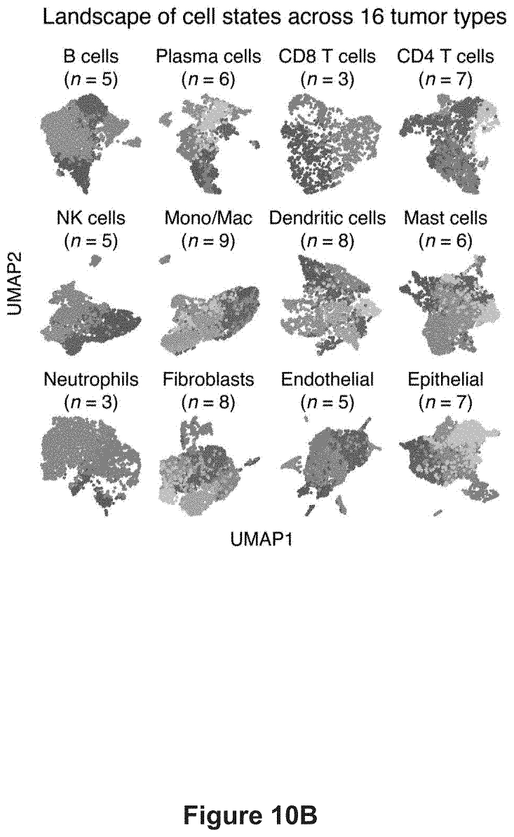

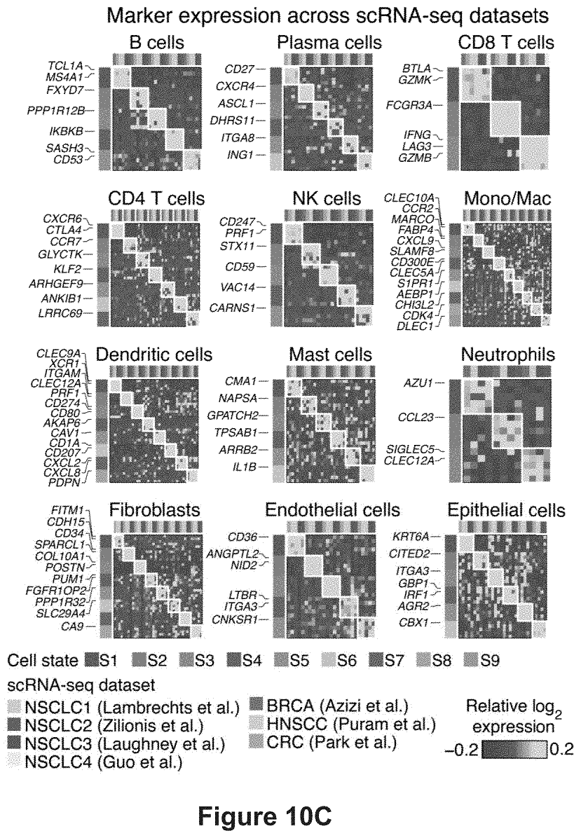

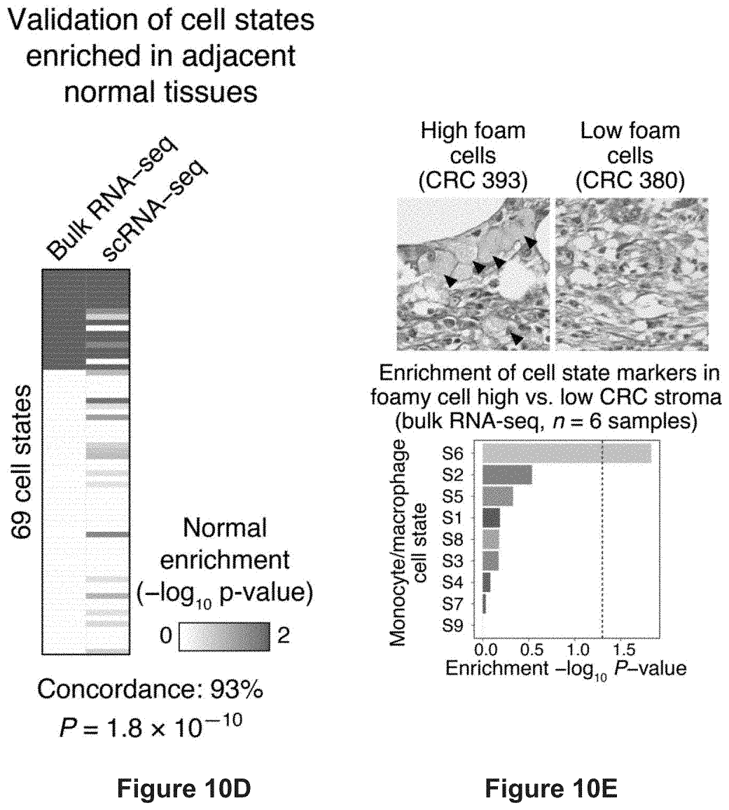

After quality control filtering, EcoTyper yielded 71 discrete cell states, ranging from 3 to 9 states per cell type (FIGS. 10A-10B). Most states were ubiquitous across carcinomas and significantly enriched in malignant tissue, highlighting key commonalities independent of tumor site. Nevertheless, many states also varied in their histological or clinical distribution. For example, multiple phenotypic programs distinguished neoplastic from adjacent normal tissues and adenocarcinomas from squamous cell carcinomas. We also observed fundamental differences with respect to cell lineage: epithelial states showed the strongest specificity for particular tumor types, followed by fibroblasts, myeloid cells (aside from neutrophils), lymphocytes, and endothelial cells.

EcoTyper implements a supervised framework for reference-guided annotation, in which cell states learned in one dataset can be identified and statistically evaluated in another. Therefore, to assess the fidelity of the 71 cell states defined by EcoTyper, we queried each state in 200,000 single-cell transcriptomes covering four types of human carcinoma: non-small cell lung cancer (NSCLC), breast cancer, colorectal cancer (CRC), and head and neck squamous cell carcinoma (HNSC). (See Guo, X., et al. (2018). Global characterization of T cells in non-small-cell lung cancer by single-cell sequencing. Nature Medicine 24, 978-985; Lambrechts, D., et al. (2018). Phenotype molding of stromal cells in the lung tumor microenvironment. Nat Med 24, 1277-1289; Laughney, A. M., et al. (2020). Regenerative lineages and immune-mediated pruning in lung cancer metastasis. Nat Med 26, 259-269; Zilionis, R., et al. (2019). Single-Cell Transcriptomics of Human and Mouse Lung Cancers Reveals Conserved Myeloid Populations across Individuals and Species. Immunity 50, 1317-1334 e1310; Azizi, E., et al. (2018). Single-Cell Map of Diverse Immune Phenotypes in the Breast Tumor Microenvironment. Cell 174, 1293-1308 e1236; Lee et al., 2020; and Puram, S. V., et al. (2017). Single-Cell Transcriptomic Analysis of Primary and Metastatic Tumor Ecosystems in Head and Neck Cancer. Cell 171, 1611-1624 e1624; the disclosures of which are hereby incorporated by reference in their entireties.) In all, 94% of cell states (67 of 71) were significantly recoverable in scRNA-seq data using reference-guided annotation coupled with permutation testing. The recovery rate remained high regardless of cell type or dataset, underscoring the robustness of our results. Moreover, we observed strikingly reproducible marker gene expression across all seven scRNA-seq tumor atlases, with a leave-one-out cross-validation rate of 90% (FIG. 10C). As an alternative approach, we tested whether states enriched in particular biological groupings (e.g., normal tissues) were recapitulated at the single-cell level. Indeed, after mapping single-cell transcriptomes to EcoTyper states, we observed significant concordance for states enriched in adjacent normal tissues, adenocarcinomas, and squamous cell carcinomas (P<0.05, Fisher's exact test; FIG. 10D). Based on these assessments, we selected 69 of 71 states for further analysis, omitting two that mapped to potential doublets in scRNA-seq data (endothelial cells state 3, fibroblasts state 7).

We next annotated each state by comparison to known transcriptional programs, prominently expressed marker genes, and states defined by previous scRNA-seq studies. Approximately two-thirds of EcoTyper states were attributable to genes or phenotypes established in prior literature. For example, without prior knowledge, EcoTyper identified tip-like endothelial cells (ANGPTL2+/NID2+) implicated in tumor neovascularization; two fibroblast states previously described in head and neck squamous cell carcinoma (CAF1 and CAF2; FIG. 10A); and canonical T cell subsets associated with pre-effector, exhaustion, and resting phenotypes (CCR7+, LAG3+, KLF2+, respectively; FIG. 10C). (See e.g., Kadomatsu, T., et al. (2014). Diverse roles of ANGPTL2 in physiology and pathophysiology. Trends in Endocrinology & Metabolism 25, 245-254; and Zhao, Q., et al. (2018). Single-Cell Transcriptome Analyses Reveal Endothelial Cell Heterogeneity in Tumors and Changes following Antiangiogenic Treatment. Cancer Research 78, 2370-2382; the disclosures of which are hereby incorporated by reference in their entireties.) EcoTyper also revealed insights into cell types with poorly understood plasticity in cancer. For example, among cells of the monocyte/macrophage lineage, which have emerging roles in cancer immunotherapy, EcoTyper reconstructed nine in vivo phenotypes with broad representation, including states consistent with pro-inflammatory monocytes (CCR2+), classical M0 macrophages (FABP4+), and M1 macrophages (CXCL9+) (FIG. 2C,E; Figure S3D; Table S4). (Feng, M., et al. (2019). Phagocytosis checkpoints as new targets for cancer immunotherapy. Nature Reviews Cancer 19, 568-586; the disclosure of which is hereby incorporated by reference in its entirety.) Four candidate subtypes of M2-like macrophages were also detectable (states 4 to 7), including states expressing known M2 marker genes such as CD209 and CD163 (state 4); S1PR1 (state 5), and CHI3L2 (state 7) (FIG. 10C). (See Murray, P. J., and Wynn, T. A. (2011). Protective and pathogenic functions of macrophage subsets. Nature Reviews Immunology 11, 723-737; Tong, L., et al. (2019). CLEC5A expressed on myeloid cells as a M2 biomarker relates to immunosuppression and decreased survival in patients with glioma. Cancer Gene Therapy; and Weichand, B., et al. (2017). 51 PR1 on tumor-associated macrophages promotes lymphangiogenesis and metastasis via NLRP3/IL-1β. Journal of Experimental Medicine 214, 2695-2713; the disclosures of which are hereby incorporated by reference in their entireties.)

Importantly, nearly one-third of EcoTyper states appeared to be novel or not previously identified by scRNA-seq surveys of human carcinomas. For example, among M2-like macrophages, we identified an AEBP1+ population (state 6) with marked similarity to foamy macrophages, a lipid-laden phenotype frequently associated with atherosclerotic plaques (Moore et al., 2013) but whose relevance across carcinomas is unclear (FIG. 10E). (See Majdalawieh, A., et al. (2006). Adipocyte enhancer-binding protein 1 is a potential novel atherogenic factor involved in macrophage cholesterol homeostasis and inflammation. Proceedings of the National Academy of Sciences 103, 2346-2351; and Moore, K. J., et al. (2013). Macrophages in atherosclerosis: a dynamic balance. Nature Reviews Immunology 13, 709-721; the disclosures of which are hereby incorporated by reference in their entireties.) To substantiate this state, we performed bulk RNA-seq of stromal cells isolated from formalin-fixed paraffin-embedded human CRC tumor biopsies with high and low foamy macrophage content (FIG. 10E). Indeed, of nine monocyte/macrophages states identified by EcoTyper, state 6 was uniquely enriched in foamy macrophage-rich stroma, corroborating our result (FIG. 10E).

Collectively, these analyses demonstrate the performance of EcoTyper and underscore its value for defining cell type-specific transcriptional programs at scales that currently exceed the practical limitations of other technologies.

### Global View of Cell-State Prognostic Associations

We and others have previously shown that cell type-specific reference profiles derived from external sources, including bulk-sorted populations and scRNA-seq data, can predict cancer clinical outcomes. However, the prognostic impact of context-dependent cellular states in human carcinoma is largely unknown. We therefore leveraged the unique output of EcoTyper to chart the prognostic landscape of 69 cell states in 15,000 tumors.

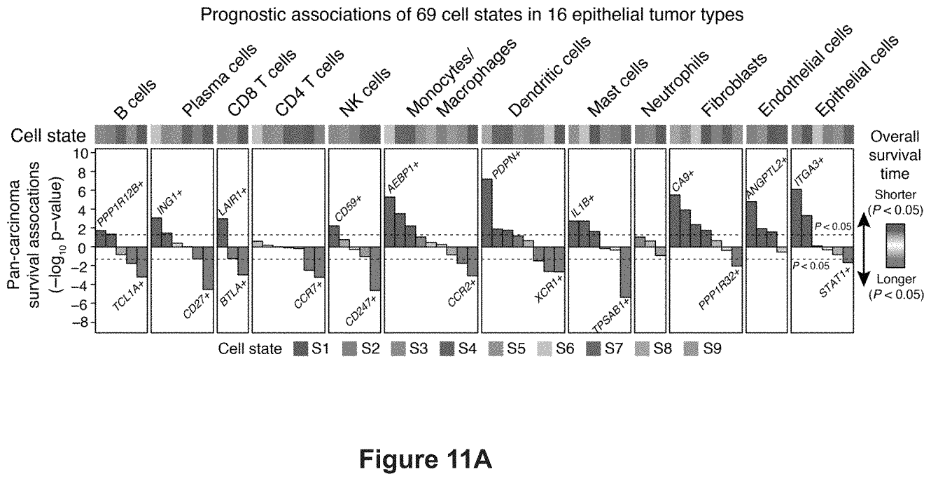

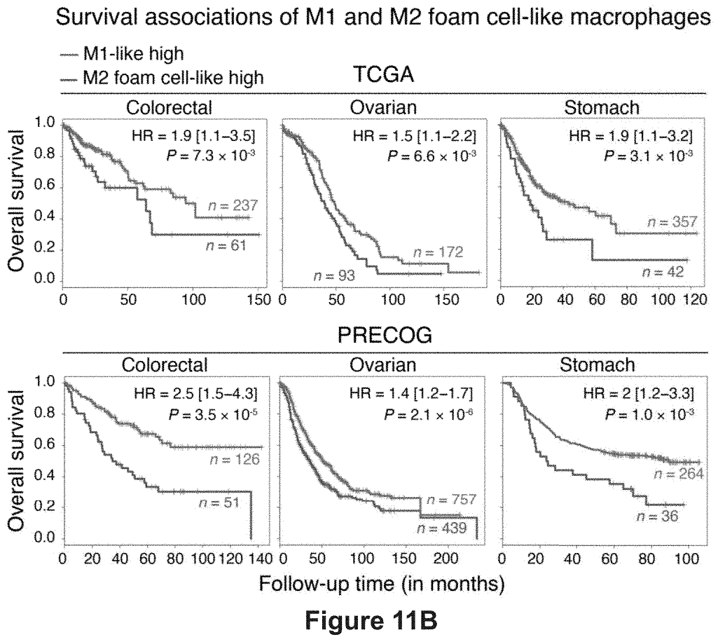

Across the 16 epithelial cancer types surveyed in our discovery cohort, the majority of cell states (39 of 69) were significantly associated with overall survival (FIG. 11A) and 49% (n=34) were significant in multivariate analyses incorporating stage, age, and sex. Global survival associations dichotomized nearly all evaluated cell types into favorable and adverse states, highlighting their biological and clinical heterogeneity (FIG. 11A). Indeed, every cell type except CD4 T cells had at least one favorable and one adverse state. For example, macrophage subsets annotated as M1 (state 3) and M2 (states 4 to 7) were associated with longer and shorter survival time, respectively, as found in prior studies (FIG. 11A). (See Mehla, K., and Singh, P. K. (2019). Metabolic Regulation of Macrophage Polarization in Cancer. Trends in Cancer 5, 822-834; the disclosure of which is hereby incorporated by reference in its entirety.) Surprisingly, among M2-like states, AEBP1+ foamy macrophages were among the top five determinants of adverse survival, suggesting that foam cells could have widespread relevance as an immunotherapeutic target in cancer (FIG. 11A). Other notable states associated with adverse risk included CA9+ fibroblasts (state 8) and POSTN+ fibroblasts (state 3), both of which have been implicated in tumor invasiveness; and pro-angiogenic tip-like endothelial cells (state 2) (FIG. 11A). (See Fiaschi, T., et al. (2013). Carbonic anhydrase IX from cancer-associated fibroblasts drives epithelial-mesenchymal transition in prostate carcinoma cells. Cell Cycle 12, 1791-1801; and Gonzalez-Gonzalez, L., and Alonso, J. (2018). Periostin: A Matricellular Protein With Multiple Functions in Cancer Development and Progression. Frontiers in Oncology 8; the disclosures of which are hereby incorporated by reference in their entireties.) Specific leukocyte populations dominated favorable outcomes across carcinomas, with leading states including naïve/central memory CD4 T cells (CCR7+), CD247+NK cells, CD27+ plasma cells, and XCR1+ cDC1-like dendritic cells, which are associated with CD8 T cell priming (FIG. 11A). (See Sanchez-Paulete, A. R., et al. (2017). Antigen cross-presentation and T-cell cross-priming in cancer immunology and immunotherapy. Annals of Oncology 28, xii44-xii55; the disclosure of which is hereby incorporated by reference in its entirety.)

To determine the generalizability of these results, we applied EcoTyper to quantitate all 69 cell states in an independent cohort of 9,062 epithelial tumor transcriptomes from PRECOG, for which overall survival data are available. State-specific survival associations across carcinomas, as measured by weighted z-scores, were highly concordant across cohorts (Pearson r=0.76, P=1.8×10⁻¹³; FIG. 11C, corroborating our findings and emphasizing the extensibility of EcoTyper to new datasets. We also observed high concordance for individual tumor types, such as colon, ovarian, and gastric cancers, for which M1 and M2 foamy-like macrophages predicted longer and shorter survival time, respectively (FIG. 11B).

### Large-Scale Reconstruction of Multicellular Communities In Vivo

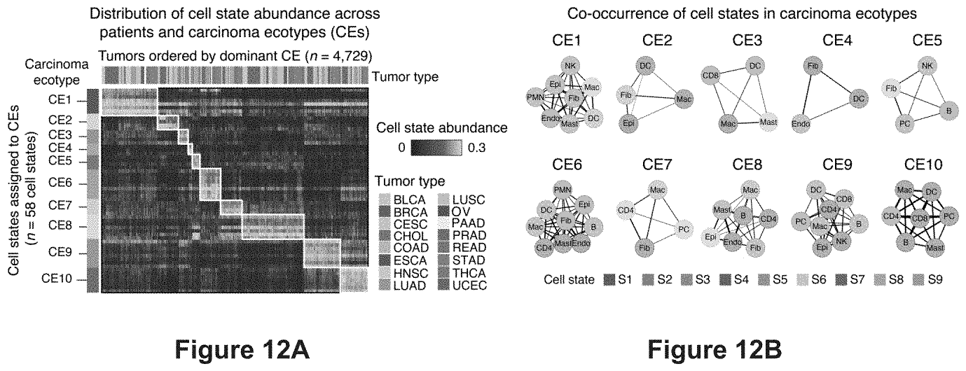

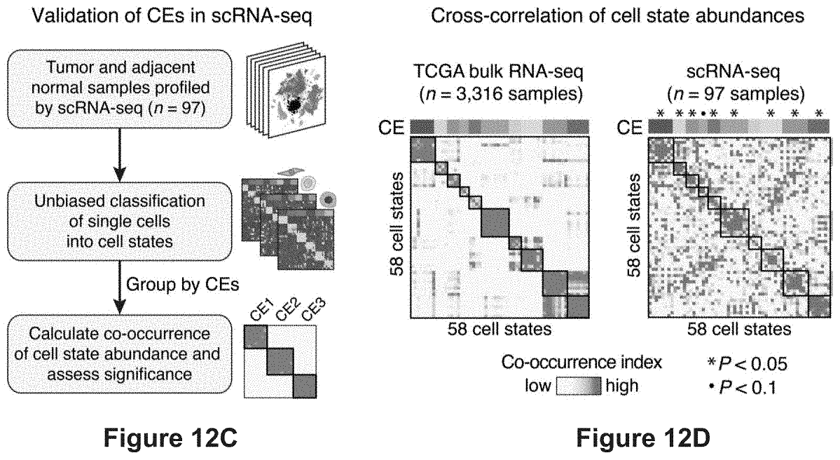

Tumors are complex ecosystems comprised of spatially and temporally-linked cell states. To determine whether EcoTyper can reconstruct multicellular ecosystems at scale, we devised a data-driven approach for clustering cell states based on patterns of co-occurrence and mutual avoidance. By applying this approach to tumor samples in the discovery cohort (69 states×5,946 tumors), we identified 10 strikingly cohesive cellular communities, which we termed carcinoma ecotypes (CEs) (FIGS. 12A-12B). CEs ranged from 3 to 9 distinct cell states per community (FIGS. 12A-12B), were robustly recovered independent of clustering approach, largely ubiquitous across human carcinomas (FIG. 12A), and highly distinct from recently described immunological subtypes in TCGA (Thorsson et al., 2018). Moreover, by aggregating across cell state abundance profiles, CE composition could be assessed in a continuous manner. (See Thorsson, V., et al. (2018). The Immune Landscape of Cancer. Immunity 48, 812-830.e814; the disclosure of which is hereby incorporated by reference in its entirety.) While nearly every tumor sample had a dominant CE (FIG. 12A), most tumors were comprised of multiple CEs, emphasizing widespread modularity in neoplastic tissue composition.

To gauge the validity of these results, we performed three technical experiments. First, we tested whether CEs are reproducible in independent datasets. By performing dimensionality reduction with UMAP on cell-state abundance profiles, we observed nearly identical community structure in >6,000 held-out epithelial tumors. Second, we tested whether CEs are enriched for cell states with interaction potential. Indeed, when compared to background expectations, 60% of CEs were significantly enriched in ligand-receptor pairs.

Given these results, we next asked whether CEs are detectable at the single-cell level. Using the scRNA-seq compendium described above, which includes ˜200 k single-cell transcriptomes encompassing 123 tumor and 49 adjacent normal specimens from four carcinomas, we assigned cells to EcoTyper states without prior knowledge of CEs (FIG. 12C). We then determined the fractional abundance of each state within each tumor/normal sample and grouped cell states into predefined CE classes. Finally, we determined whether states assigned to the same CE are more strongly co-associated than expected by random chance (FIG. 12C). In all, 80% of CEs were significantly detectable in scRNA-seq data (P<0.05). Moreover, 90% were detectable at P<0.1 (FIG. 12D). This result was striking given potential confounding factors in scRNA-seq data that could obscure CE detection, including modest sample sizes, low cell numbers per sample, and sparsity in gene expression. As an alternative approach, we determined the joint probability of obtaining 10 CEs with equally strong co-associations by random chance. Relative to background expectations, the probability of obtaining our original result by random chance was less than 1 in 1,000,000 (P<10-6).

Taken together, these data validate our approach, identify distinct multicellular communities in bulk and single-cell expression data, and nominate CEs as fundamental units of cellular organization across human carcinomas (FIG. 12D).

### Carcinoma Ecotype Characteristics in 6 k Normal and Neoplastic Tissue Specimens

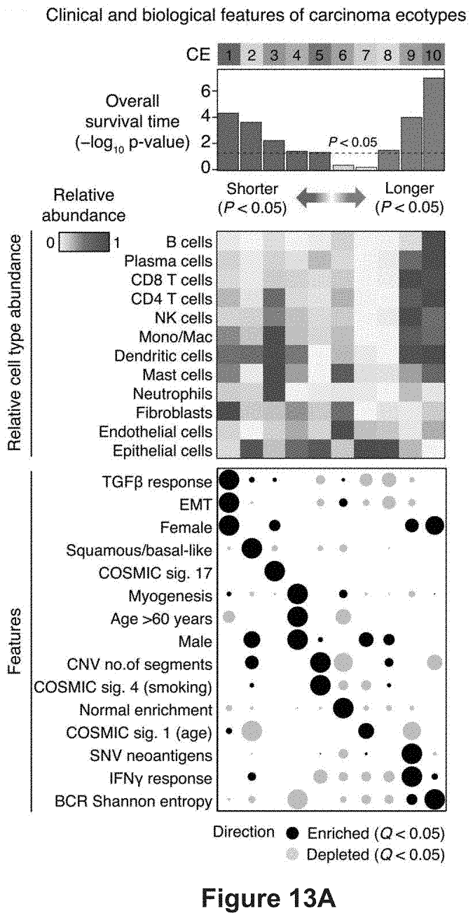

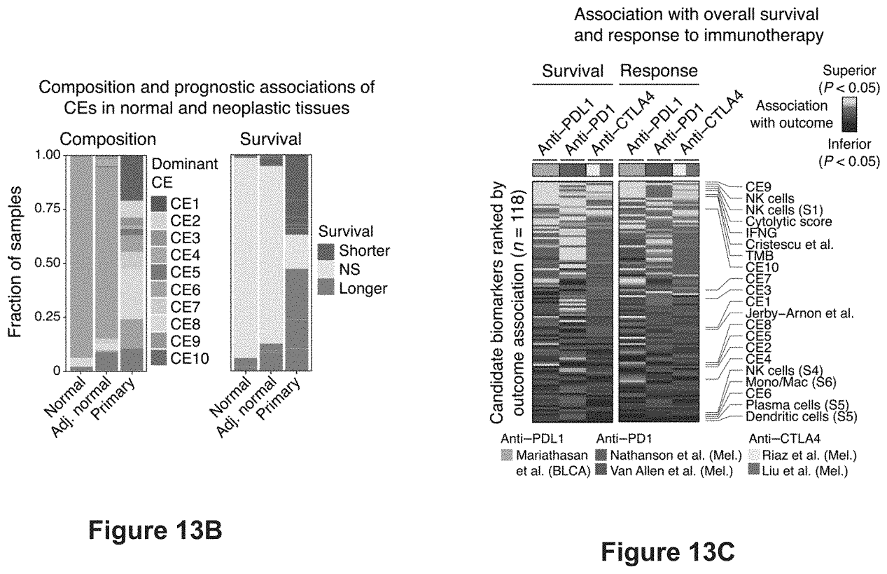

Having identified ten dominant multicellular ecosystems in carcinoma, we next explored their cellular, genomic, and clinical characteristics (FIG. 13A). Across the discovery cohort, eight CEs were significantly prognostic in univariate models, and five remained significant after multivariate adjustments for stage, age, and sex (FIG. 13A). CE1- and CE2-high tumors were lymphocyte-deficient, strongly linked to higher risk of death, and broadly distinguished by elevated levels of POSTN+ fibroblasts and basal-like epithelial cells, respectively (FIG. 13A; FIG. 12B). CE3-high tumors, predictive of worse survival outcome, were myeloid-enriched, microsatellite instability (MSI) high, and associated with COSMIC mutational process 17, a signature found in esophageal and gastric cancers, and linked, at least in part, to gastric reflux. (See Christensen, S., et al. (2019). 5-Fluorouracil treatment induces characteristic T>G mutations in human cancer. Nature Communications 10, 4571; the disclosure of which is hereby incorporated by reference in its entirety.) CE4-high tumors were associated with myogenesis and males over 60 years of age, whereas CE5- through CE8-high tumors were enriched for smoking-related mutations, normal tissue, age-related mutations, and moderately favorable outcomes, respectively. Finally, CE9- and CE10-high tumors were proinflammatory (i.e., leukocyte rich), strongly associated with longer overall survival, and characterized by higher immunoreactivity, including IFN-γ signaling, and higher B cell content, respectively. Notably, two CEs were present at similar frequencies in tumor and adjacent normal tissues but deficient in healthy tissues (CE4, CE10), reflecting a potential field effect. Others, with the exception of CE6, were largely specific to neoplastic tissue (FIG. 13B).

### Multicellular Prediction of Immunotherapy Response

Since each carcinoma ecotype integrates contributions from multiple cell states, we reasoned that CE profiling might have the potential to more accurately predict clinical outcomes than any individual state alone. To test this possibility, we compiled tumor expression data from 571 patients with advanced metastatic disease prior to receiving immune checkpoint blockade with anti-PDL1 (urothelial carcinoma), anti-PD1 (melanoma), or anti-CTLA4 (melanoma) monotherapy. We included metastatic melanoma in this analysis as most non-epithelial cell states reliably generalized to this disease. To quantify performance, we evaluated continuous associations with overall survival and binary associations with immunotherapy response. CE9, which is characterized by IFN-γ signaling, outperformed other CEs for predicting superior outcomes across therapy types and outcome measures (FIG. 13C). We also compared CE profiling to 108 candidate biomarkers, including 69 cell states quantitated by EcoTyper, 25 parental populations enumerated by CIBERSORTx, a cytolytic score, tumor mutational burden (TMB), and two published signatures of ICI response. (See e.g., Cristescu, R., et al. (2018). Pan-tumor genomic biomarkers for PD-1 checkpoint blockade-based immunotherapy. Science 362, eaar3593; the disclosure of which is hereby incorporated by reference in its entirety.) Surprisingly, CE9 surpassed all other measures including those designed to predict ICI response (FIG. 13C). These data suggest that multicellular communities, even in the absence of optimization, can capture biological signal with superior predictive value.

### Spatiotemporal Dynamics of Proinflammatory Communities

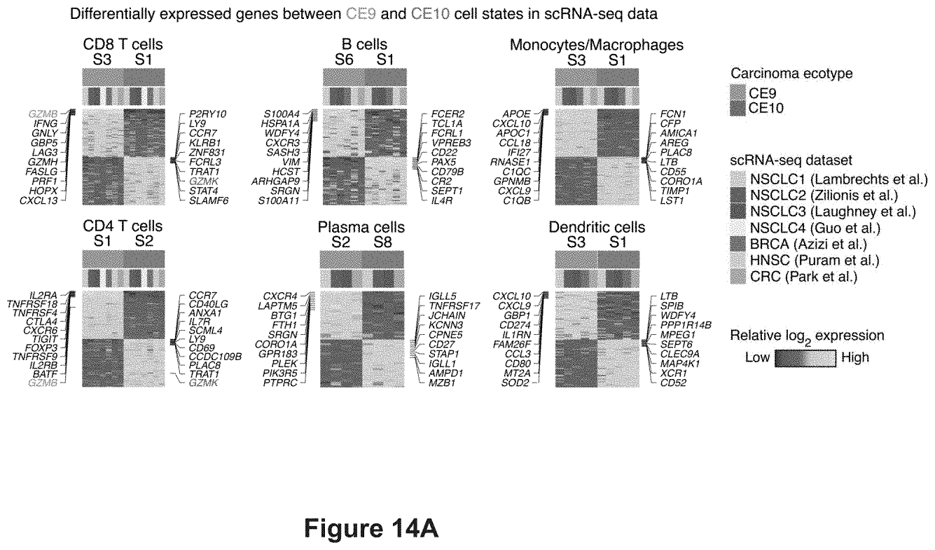

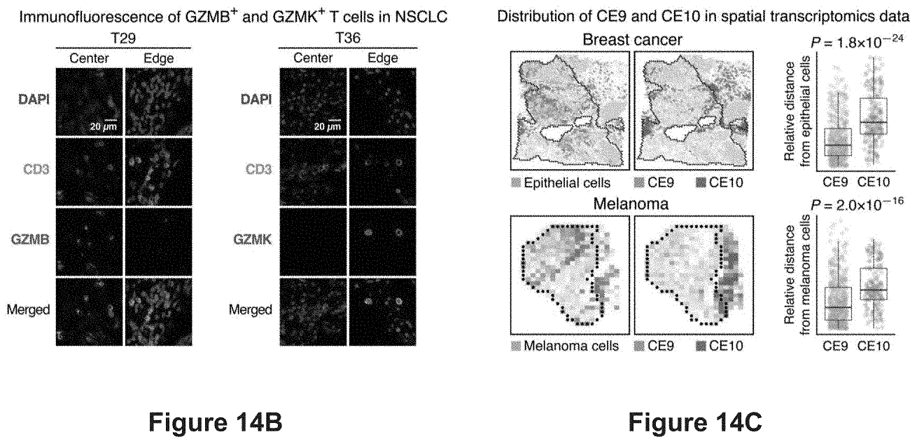

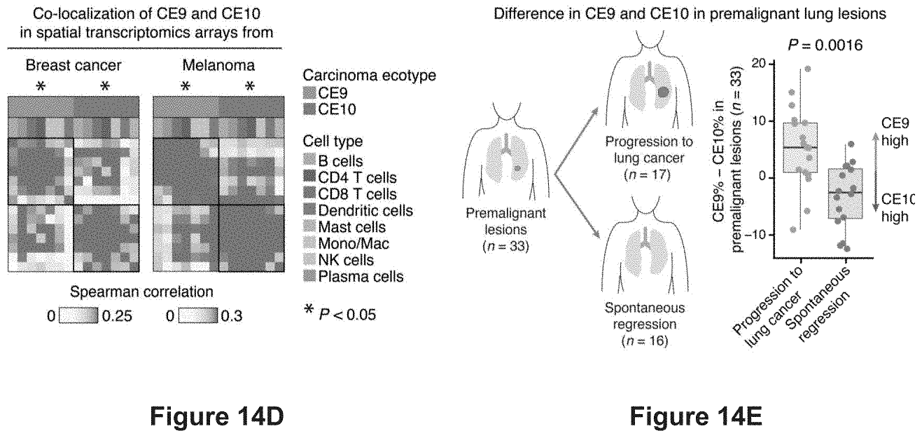

We next sought to determine whether carcinoma ecotypes show distinct patterns of spatial organization. To do so, we focused on CE9 and CE10, two proinflammatory communities with canonical T cell states and favorable overall survival, but otherwise disparate genomic and cellular features. CE9-T cells upregulate activation and immunoregulatory genes, including markers of exhaustion, consistent with the association of CE9 with response to ICI (e.g., LAG3, IFNG, HAVCR2, CTLA4). In contrast, CE10-T cells express markers of naïve and central memory cells (e.g., CCR7) (FIG. 14A). Although such differences are well-documented in tumor-associated T cells, their precise cellular communities have not been previously established. (See e.g., Oh, D. Y., et al. (2020). Intratumoral CD4+ T Cells Mediate Anti-tumor Cytotoxicity in Human Bladder Cancer. Cell; and Zheng, C., Zheng, L., Yoo, J.-K., Guo, H., Zhang, Y., Guo, X., Kang, B., Hu, R., Huang, J. Y., Zhang, Q., et al. (2017). Landscape of Infiltrating T Cells in Liver Cancer Revealed by Single-Cell Sequencing. Cell 169, 1342-1356.e1316; the disclosures of which are hereby incorporated by reference in their entireties.) With EcoTyper, we found that CE9-T cells strongly co-occur with six cellular states, including ones resembling M1 macrophages, mature immunogenic dendritic cells, and activated B cells. Conversely, CE10-T cells co-occur with five cellular states, including those consistent with pro-inflammatory monocytes, cDC1 dendritic cells, and resting B cells (FIGS. 12B, 14A). These results were confirmed across seven scRNA-seq datasets via reference-guided annotation, reinforcing the notion that specific phenotypes preferentially co-occur as multicellular assemblies in the tumor microenvironment (FIG. 14A; FIG. 12D).

To check whether CE-specific phenotypes are spatially distinct, we first performed multicolor immunofluorescence staining for GZMB+ and GZMK+, (FIG. 14B), which respectively mark CE9 and CE10-T cells (FIG. 14A). In cancer, GZMB and GZMK have been observed to distinguish activated effector and transitional effector memory T cells, respectively, however, to our knowledge, single-cell localization patterns of GZMK+ T cells in human tumors have not been previously described. (See e.g., Li, H., et al. (2019). Dysfunctional CD8 T Cells Form a Proliferative, Dynamically Regulated Compartment within Human Melanoma. Cell 176, 775-789.e718; the disclosure of which is hereby incorporated by reference in its entirety.) We applied EcoTyper to 23 bulk tumor transcriptomes from patients with NSCLC and selected four specimens with distinct CE9 and CE10 composition. Multiplexed staining of these specimens verified EcoTyper predictions. Additionally, while GZMB+ T cells were localized to the tumor core, consistent with a link between chronic antigen stimulation and T cell exhaustion, GZMK+ T cells were excluded, instead localizing at the periphery (FIG. 14B). (See Wherry, E. J., and Kurachi, M. (2015). Molecular and cellular insights into T cell exhaustion. Nature Reviews Immunology 15, 486-499; the disclosure of which is hereby incorporated by reference in its entirety.)

To extend our analysis to additional cell types and malignancies, we next turned to in situ spatially-barcoded microarray data generated from fresh/frozen breast (10× Visium) and melanoma tumor sections. (See e.g., Thrane, K., et al. (2018). Spatially Resolved Transcriptomics Enables Dissection of Genetic Heterogeneity in Stage III Cutaneous Malignant Melanoma. Cancer Res 78, 5970-5979; the disclosure of which is hereby incorporated by reference in its entirety.) By aggregating across all states within each ecotype, we found that CE9 was detectable at the core and invasive margin of the tumor whereas CE10 was generally localized along the periphery of the same tumor mass (FIG. 14C), consistent with immunofluorescence imaging (FIG. 14B). These spatial differences were highly significant with regard to distance from tumor cells (P<10⁻⁵; FIG. 14C) and were independent of tumor type. Moreover, by evaluating cell-state co-association patterns across distinct spatial regions, we found that cell states within CE9 and CE10 generally colocalize in a CE-specific manner regardless of developmental lineage (FIG. 14D). Thus, despite the fact that CE9 and CE10 were defined agnostic to spatial context, they occur in spatially-distinct cellular neighborhoods.

Given these results, coupled with the observation that CE10 is present in adjacent normal tissue and, to a much lesser extent, in healthy tissue (FIG. 13B), we hypothesized that CE10 precedes CE9 during early tumor development. Consistent with this hypothesis, we found that CE10 was more prevalent than CE9 during the earliest stages of squamous cell lung carcinogenesis, whereas in malignant tissue, CE9 was more prevalent than CE10. Moreover, in precancerous lesions of lung squamous cell carcinoma collected from 33 subjects with known outcomes, higher relative levels of CE10 were significantly associated with spontaneous regression whereas higher relative levels of CE9 predicted progression to invasive cancer (area under the curve=0.82; P=0.001; two-sided Wilcoxon rank sum test; FIG. 14E). (See Teixeira, V. H., et al. (2019). Deciphering the genomic, epigenomic, and transcriptomic landscapes of pre-invasive lung cancer lesions. Nature Medicine 25, 517-525; the disclosure of which is hereby incorporated by reference in its entirety.) Together these data further validate our approach, link CE dynamics to early lung cancer development, and provide a platform to systematically interrogate the diagnostic and therapeutic potential of tumor cellular ecosystems.

DISCUSSION: In this study, we describe EcoTyper as a new system for decoding cell states and multicellular communities from primary tissue transcriptomes. EcoTyper is distinguished from related technologies in several important ways: First, by imputing cellular heterogeneity directly from RNA profiles of intact tissue biopsies, EcoTyper avoids distortions induced by physical cell isolation, does not require antibodies or preselection of phenotypic markers, and is applicable to fresh, frozen, and fixed specimens. Second, unlike previous computational approaches, EcoTyper can accurately resolve transcriptional states from multiple cell types (>10), assemble them into multicellular communities, quantify their relative composition, and query them across diverse expression datasets and platforms. Although EcoTyper was applied across 16 carcinomas in this work, it is generalizable to any tissue type and disease state for which suitable expression data are available.

Although recent studies have revealed critical insights into tumor cellular communities using multiplexed imaging, these studies focused on single tumor types using a limited number of predefined phenotypic markers (<60). (See e.g., Keren, L., Bosse, M., Thompson, S., Risom, T., Vijayaragavan, K., McCaffrey, E., Marquez, D., Angoshtari, R., Greenwald, N. F., Fienberg, H., et al. (2019). MIBI-TOF: A multiplexed imaging platform relates cellular phenotypes and tissue structure. Sci Adv 5, eaax5851; the disclosure of which is hereby incorporated by reference in its entirety.) By deploying EcoTyper to analyze 16 types of human carcinoma spanning nearly 6,000 bulk tumor transcriptomes, we uncovered 69 transcriptionally-defined cell states and 10 previously unknown multicellular communities in a marker-agnostic manner. Our study is the first, to our knowledge, to characterize multicellular communities at the transcriptional level across thousands of solid tumors, corroborate them in single-cell RNA-sequencing data, and assess their associations with ICI response and early cancer development. These data and associated analytical tools provide new opportunities for the development of diagnostic and therapeutic strategies that rely upon knowledge of tumor-associated cell states and their patterns of multicellular interaction.

Despite the promise of EcoTyper, several challenges remain. For example, EcoTyper requires reference profiles that distinguish major cell types within a tissue type of interest. Given the rapid pace of single-cell sequencing efforts (e.g., Human Tumor Atlas Network), this requirement is unlikely to be a major hurdle for most applications. (See Rozenblatt-Rosen, O., et al. (2020). The Human Tumor Atlas Network: Charting Tumor Transitions across Space and Time at Single-Cell Resolution. Cell 181, 236-249; the disclosure of which is hereby incorporated by reference in its entirety.) Second, not all cell states are resolvable by EcoTyper, either because they fall beneath the lower limit of detection (˜0.1%), are not definable from the genes imputed by CIBERSORTx, or exhibit nearly perfect covariance with other cell states. Methodological improvements to overcome these issues are underway.

In summary, we demonstrate how cell states and multicellular communities can be profiled from bulk tissue transcriptomes, recovered in expression datasets independent of platform, related to immunotherapy response, and tracked across space and developmental time. Our approach is accurate, highly complementary to existing single-cell assays, and has significant potential for generating experimentally-testable hypotheses. Given its unique capabilities, we anticipate that EcoTyper will prove useful for reconstructing cellular community structure at high resolution and massive scale in health and disease.

### DOCTRINE OF EQUIVALENTS

Although the invention has been described in detail with particular reference to these preferred embodiments, other embodiments can achieve the same results. Variations and modifications of the present invention will be obvious to those skilled in the art and it is intended to cover all such modifications and equivalents. The entire disclosures of all references, applications, patents, and publications cited above, and of the corresponding application(s), are hereby incorporated by reference.

What is claimed is:

1. A method for treating an individual for a tumor, comprising:
obtaining gene expression data from a tumor obtained from an individual;
characterizing a tumor ecosystem for the tumor based on the gene expression data, wherein the tumor ecosystem is comprised of spatially and temporally-linked cell states;
identifying an efficacious treatment for the tumor based on clinical treatment data, wherein the clinical treatment data identifies at least one treatment shown to be efficacious for a tumor exhibiting the tumor ecosystem; and
treating the individual with the efficacious treatment for the tumor.

2. The method of claim 1, wherein the characterizing a tumor ecosystem step comprises:
purifying a gene expression profile of cell types within the tumor;
identifying at least one cell state in the tumor based on the gene expression profiles; and
identifying the tumor ecosystem based on the at least one cell state.

3. The method of claim 2, wherein the identifying the tumor ecosystem step comprises using a trained negative matrix factorization (NMF) model to identify the tumor ecosystem.

4. The method of claim 3, wherein the NMF model is trained by:
obtaining cellular expression data from a plurality of samples from one or more tissue types;
purifying gene expression profiles of cell types within plurality of samples based on the cellular expression data;
identifying cell states of the cell types by clustering cell type-specific gene expression profiles; and
classifying the plurality of samples into tumor ecosystem subtypes by identifying cell states that co-occur in the same sample.

5. The method of claim 4, wherein the purifying step uses a digital cytometry algorithm for to purify the gene expression profiles.

6. The method of claim 5, wherein the digital cytometry algorithm is CIBERSORTx.

7. The method of claim 4, wherein the one or more tissue types include at least one cancer or tumor.

8. The method of claim 7, wherein the at least one cancer or tumor is selected from the group consisting of: lymphomas and carcinomas.

9. The method of claim 7, wherein the at least one cancer or tumor is selected from the group consisting of: diffuse large B cell lymphoma, -small cell lung cancer, breast cancer, colorectal cancer, and head and neck squamous cell carcinoma.

10. The method of claim 4, wherein the cellular expression data is obtained from single cell RNA sequencing.

11. The method of claim 4, wherein the NMF model is employed via Kullback-Leibler divergence minimization.

12. The method of claim 4, wherein the identifying cell states calculate a cophenetic coefficient for a range of cluster numbers as part of clustering.

13. The method of claim 4, wherein the clustering further comprises filtering to remove low quality cell states.

14. The method of claim 13, wherein the filter removes cell states with fewer than 10 genes.

15. The method of claim 13, wherein the filter removes cell states with low levels of expression.

16. The method of claim 4, wherein the NMF model training further comprises updating the NMF model by iteratively updating the model until convergence.

17. The method of claim 1, wherein the at least one treatment is selected from chemotherapeutics, immunotherapeutics, radiation, and combinations thereof.

18. The method of claim 1, further comprising obtaining a tumor sample or a cancer sample from an individual, wherein the gene expression data is obtained from the tumor sample or the cancer sample.

19. The method of claim 18, wherein the tumor sample or the cancer sample is obtained from a biopsy.

20. The method of claim 1, wherein the gene expression data is obtained from RNA sequencing, single cell RNA sequencing, or a microarray.
